(19) United States
(12) Patent Application Publication
(10) Pub. No.: US 2026/0007515 A1
(43) Pub. Date: Jan. 8, 2026

(54) PROSTHETIC MEDICAL DEVICE DELIVERY ASSEMBLY

(71) Applicant: EDWARDS LIFESCIENCES CORPORATION, Irvine, CA (US)

(72) Inventors: Kurt Kelly Reed, Costa Mesa, CA (US); Kevin Gantz, Laguna Beach, CA (US); Timothy Lee Humphrey, Anaheim, CA (US); Julian Lo Chiang, JR., Montclair, CA (US)

(21) Appl. No.: 19/326,564

(22) Filed: Sep. 11, 2025

Related U.S. Application Data
(63) Continuation of application No. PCT/US2024/017006, filed on Feb. 23, 2024.
(60) Provisional application No. 63/489,832, filed on Mar. 13, 2023.

(51) Int. Cl. A61F 2/24

(52) U.S. Cl. CPC A61F 2/2427

(57) ABSTRACT

A support can be configured to stabilize a delivery apparatus comprising three independently actuatable shafts extending in an axial direction. The support can comprise a track extending in the axial direction, a first cart coupled to the track and configured to couple to a first shaft, and a second cart coupled to the track and configured to couple to a second shaft. A rack can be fixedly coupled to the first cart and coupled to the second cart. The second cart can comprise a locking assembly actuatable between a first mode and a second mode. In the first mode, the second cart is slidingly coupled to the track and is fixedly coupled to the rack. In the second mode, the second cart is fixedly coupled to the track and is slidingly coupled to the rack.

### CROSS REFERENCE TO RELATED APPLICATION

This application is a continuation of PCT patent application no. PCT/US2024/017006, filed on Feb. 23, 2024, which application claims the benefit of U.S. Provisional Patent Application No. 63/489,832, filed Mar. 13, 2023, each of these applications being incorporated herein in its entirety by this specific reference.

### FIELD

The present disclosure relates to delivery assemblies for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (such as stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (such as through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

A docking device delivery system can be used to deliver a prosthetic medical device, such as a docking device used in conjunction with the prosthetic heart valve described above.

The docking device can be positioned at the implantation site by the docking device delivery system to provide for better sealing between the implantation site and the prosthetic heart valve.

### SUMMARY

The foregoing and other objects, features, and advantages of the invention will become more apparent from the following detailed description, which proceeds with reference to the accompanying figures.

Described herein are prosthetic heart valves, delivery apparatuses, delivery systems, and methods for implanting prosthetic heart valves. The disclosed prosthetic heart valves, delivery apparatuses, assemblies, and methods can, for example, can provide for improved positioning of a docking device for use with a prosthetic heart valve. The docking device can be positioned using a docking device delivery apparatus comprising three independently actuatable shafts. The docking device delivery apparatus can be coupled to a stabilizer assembly that allows for improved positioning of the docking device by better stabilizing the docking device delivery apparatus during a docking device implantation procedure. As such, the devices and methods disclosed herein can, among other things, overcome one or more of the deficiencies of typical prosthetic heart valves and their delivery apparatuses and delivery systems.

An assembly for implanting a prosthetic medical device can comprise a handle and one or more shafts coupled to the handle.

In some examples, the assembly can comprise three shafts.

In some examples, the one or more shafts can be independently actuatable in an axial direction.

In some examples, the assembly can further comprise a stabilizer assembly for stabilizing the one or more shafts.

In some examples, the stabilizer assembly can comprise a track extending in the axial direction, a first cart, and a second cart, wherein the first cart and the second cart are configured to slidingly couple to the track, the first cart is configured to couple to a first one of the one or more shafts, and the second cart is configured to couple to a second one of the one or more shafts.

In some examples, the stabilizer assembly can further comprise a rack gear fixedly coupled to the first cart and slidingly coupled to the second cart.

In some examples, the second cart can be configured to slide along both the track and the rack gear.

In some examples, the second cart can comprise a locking assembly configured to selectively engage the track and the rack gear.

In some examples, the locking assembly can be actuatable between a first mode and a second mode, wherein the locking assembly is configured to engage the rack gear and disengage the track in the first mode, and wherein the locking assembly is configured to disengage the rack gear and engage the track in the second mode.

In some examples, the locking assembly can comprise a track lock configured to engage the track and a rack gear lock configured to engage the rack gear.

In some examples, the locking assembly can further comprise a rotatable knob operably coupled to both the track lock and the rack gear lock, wherein turning the knob actuates both the track lock and the rack gear lock.

In some examples, the track can comprise a unitary structure.

In some examples, the track can comprise an extruded structure.

In some examples, the track can comprise a first rail having a first height and a second rail having a second height, wherein the first height is not equal to the second height.

In some examples, a docking device delivery assembly can comprise a delivery apparatus and a support for the delivery apparatus. The delivery apparatus can comprise a first shaft extending in a distal direction and a second shaft coaxial with the first shaft. The support can comprise a track extending in an axial direction; a first cart slidingly coupled to the track, wherein the first cart is configured to couple to the first shaft of the delivery apparatus; a guide member fixedly coupled to the first cart; and a second cart actuatable between a first mode and a second mode, wherein the second cart is configured to couple to the second shaft, in the first mode, the second cart is slidingly coupled to the track and is fixedly coupled to the guide member, and in the second mode, the second cart is fixedly coupled to the track and is slidingly coupled to the guide member.

In some examples, a support for use with a delivery apparatus can comprise a track extending in an axial direction; a first cart configured to slide along the track and couple to a first portion of the delivery apparatus; a guide rail fixedly coupled to an axial end portion of the first cart and extending in the axial direction; and a second cart configured to slide along the track and couple to a second portion of the delivery apparatus. The second cart can comprise a locking assembly actuatable between a first mode and a second mode, wherein in the first mode, the locking assembly engages the guide rail and disengages the track, and in the second mode, the locking assembly disengages the guide rail and engages the track.

In some examples, a support assembly can comprise a rack gear comprising a first axial end portion and a second axial end portion; a first cart fixedly coupled to the first axial end portion; a second cart is configured to slide along the rack gear between the first axial end portion and the second axial end portion; and a locking assembly. The locking assembly can comprise a lock configured to lock the second cart to the rack gear and a rotatable knob configured to actuate the locking assembly.

In some examples, a hub assembly support for a delivery apparatus can comprise a first post configured to slidingly couple to a track extending in an axial direction; a guide rail fixedly coupled to an axial end portion of the first post, wherein the guide rail extends from the axial end portion of the first post in the axial direction; and a second post slidingly coupled to both the track and the guide rail.

In some examples, a method of implanting a prosthetic medical device can comprise coupling a delivery apparatus to a stabilizer assembly. The delivery apparatus can comprise first and second coaxial shafts, wherein the first and second coaxial shafts are independently actuatable. The stabilizer assembly can comprise a rail oriented in an axial direction; a first post slidably couple to the rail, wherein the first shaft is configured to couple to the first post; a second post slidably coupled to the rail, wherein the second shaft is configured to couple to the second post; a guide member fixedly coupled to the first post and slidably coupled to the second post; and a locking assembly disposed on the second post and configured to selectively engage the rail and the guide member. The method can further comprise sliding the first post and the second post in unison in a distal direction along the rail; actuating the locking assembly to engage the rail and disengage the guide member; and sliding the first post in the proximal direction relative to the second post.

In some examples, an assembly can comprise one or more of the components recited in Examples 1-46 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

The above method(s) can be performed on a living animal or on a simulation, such as on a cadaver, cadaver heart, anthropomorphic ghost, simulator (for example, with body parts, heart, tissue, etc. being simulated).

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 schematically illustrates a stage in a docking device implantation procedure where a guide catheter is inserted into a blood vessel of a patient and navigated through the blood vessel and into a heart of the patient, according to one example.

FIG. 6 schematically illustrates another stage in the example docking device implantation procedure where a distal end portion of a docking device delivery apparatus is advanced from the guide catheter and into a left ventricle of the heart.

FIG. 7 schematically illustrates another stage in the example docking device implantation procedure where the distal end portion of the docking device delivery apparatus is coiled around a plurality of leaflets of the heart.

FIG. 8 schematically illustrates an optional stage in the example docking device implantation procedure where a radius of curvature of the distal end portion of the docking device delivery apparatus is increased to encircle the chordae tendineae of the heart in a variable encircling turn.

FIG. 9 schematically illustrates another optional stage in the example docking device implantation procedure where a sleeve shaft of the docking device delivery apparatus is retracted in a proximal direction to unsheathe a guard member of a docking device.

FIG. 10 schematically illustrates another optional stage in the example docking device implantation procedure where the sleeve shaft is advanced in the distal direction to axially foreshorten the guard member.

FIG. 11 schematically illustrates another stage in the example mitral valve replacement procedure where the docking device delivery apparatus is decoupled from the docking device.

FIG. 12 is a perspective view of a docking device delivery assembly configured for use during the docking device implantation procedure of FIGS. 5-11, according to one example.

FIG. 13 is a side view of a delivery apparatus for use with the docking device delivery assembly of FIG. 12, according to one example.

FIG. 14 is a cross-sectional view of a stabilizer track of the docking device delivery assembly of FIG. 12, according to one example.

FIG. 15 is a perspective view of a hub assembly support for use with the docking device delivery assembly of FIG. 12, according to one example.

FIG. 16A is a cross-sectional view of the hub assembly support of FIG. 15 in a first mode, according to one example.

FIG. 16B is a cross-sectional view of the hub assembly support of FIG. 15 in a second mode, according to one example.

FIGS. 17A-17E are side views of the docking device delivery assembly during the docking device implantation procedure of FIGS. 5-11, according to one example.

FIG. 18 is a cross-sectional view of a stabilizer track of the docking device delivery assembly of FIG. 12, according to a second example.

FIG. 19 is a perspective view of a hub assembly support, according to a second example.

FIG. 20 is a side view of a guide catheter for use with the docking device delivery system of FIG. 12, according to one example.

FIG. 21 is a side view of the guide catheter and the docking device delivery apparatus, according to one example.

FIG. 22 is a side view of the guide catheter and a docking device delivery apparatus, according to a second example.

FIG. 23 is a perspective view of a docking device for use with the docking device delivery system of FIG. 12, according to one example.

FIG. 24 is a perspective view of a prosthetic heart valve delivery apparatus, according to one example.

FIG. 25 is a perspective view of a prosthetic heart valve configured for use with the prosthetic heart valve delivery apparatus of FIG. 24, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (such as out of the patient's body), while distal motion of the device is motion of the device away from the user and toward the implantation site (such as into the patient's body). The terms “longitudinal” and “axial” refer to an axis extending in the proximal and distal directions, unless otherwise expressly defined.

The terms “lateral” and “radial” refer to an axis perpendicular to the longitudinal axis. When referring to a “lateral” direction with respect to a stabilizer assembly for a docking device delivery system, the term “lateral” refers to an axis that perpendicular to the longitudinal axis and parallel with a plane defined by a stabilizer track of the stabilizer assembly.

As used herein, “e.g.” means “for example,” and “i.e.” means “that is.”

### Introduction to the Disclosed Technology

Disclosed herein are examples of a delivery system that can be used to navigate a subject's vasculature to deliver a prosthetic medical device (such as a docking device used in conjunction with a prosthetic heart valve), tools, agents, or other therapy to a target implantation site within the body of the subject.

In connection therewith, various systems are described herein that, in some examples, can stabilize and actuate various components of the delivery system to better improve the positioning of the prosthetic.

The delivery system can comprise a plurality of shafts that can be independently actuated relative to one another. In some examples, the delivery system can comprise a delivery shaft comprising a delivery shaft lumen, a sleeve shaft disposed within the delivery shaft lumen and comprising a sleeve shaft lumen, and a pusher shaft disposed within the sleeve shaft lumen. The prosthetic can be positioned at the target implantation site by actuating the pusher shaft relative to the delivery shaft and the sleeve shaft.

Disclosed herein are exemplary devices and/or methods that can, among other things, make it easier to actuate (for example, axially move) one or more components of a delivery system relative to one or more other components of the delivery system.

### Examples of the Disclosed Technology

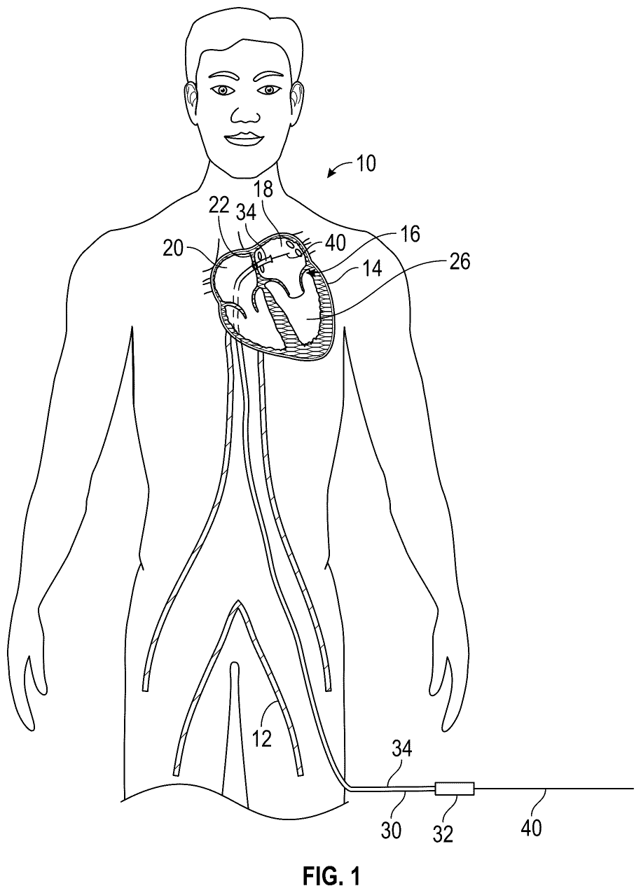

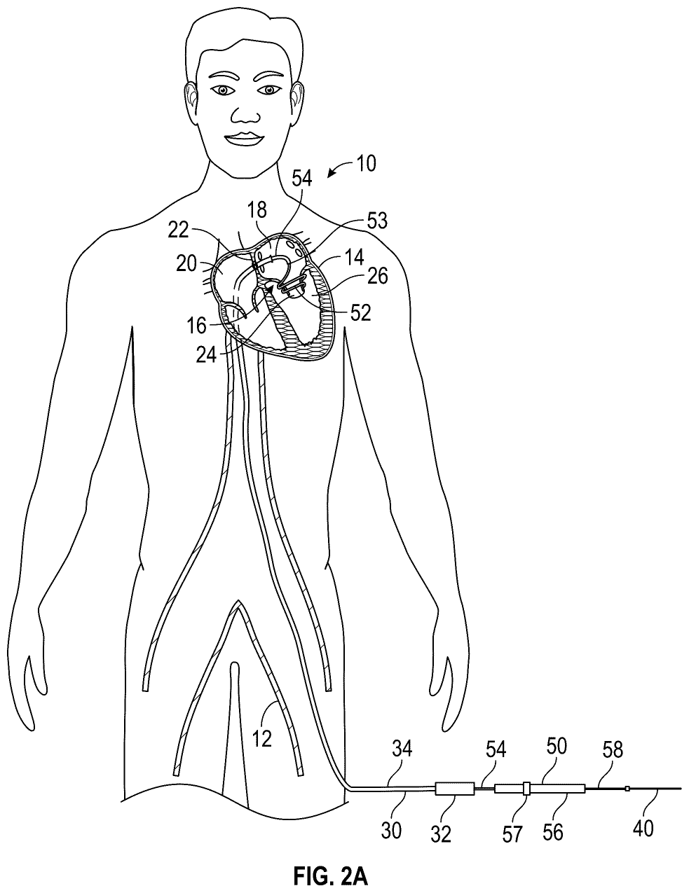

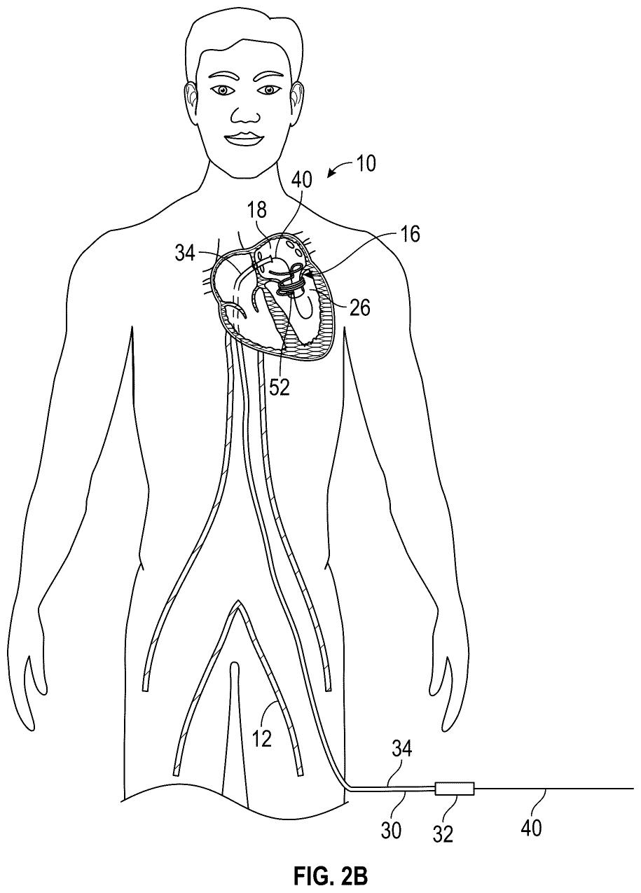

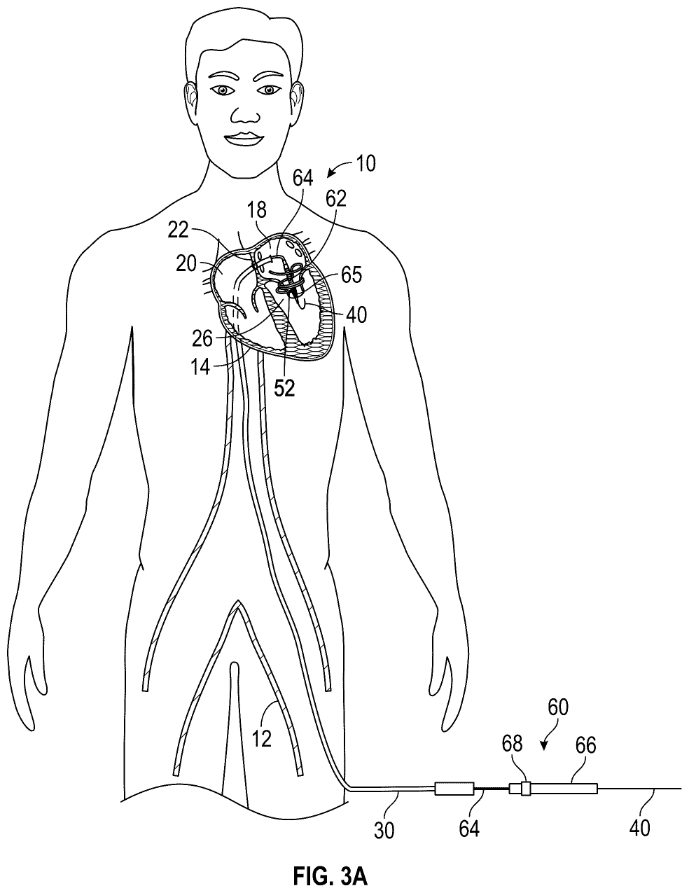

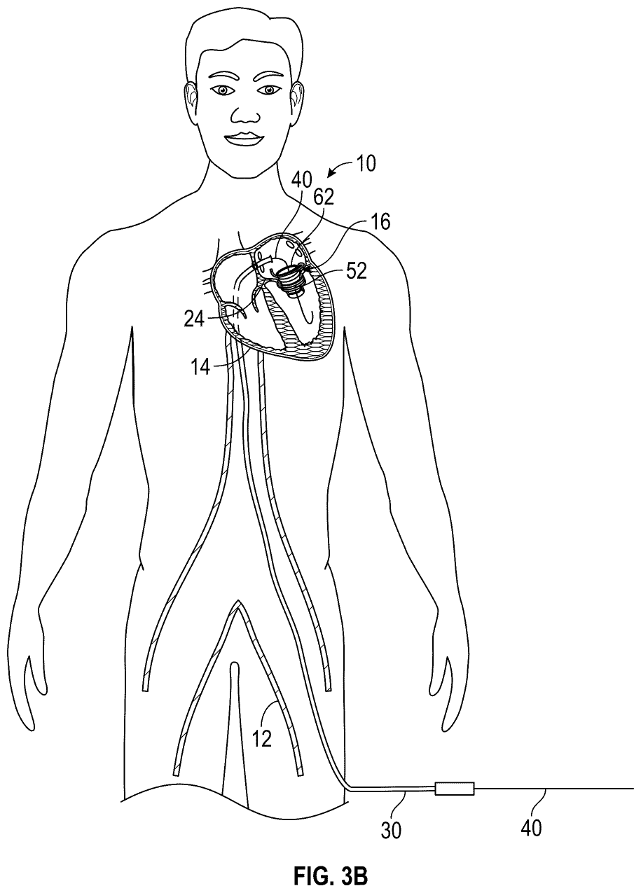

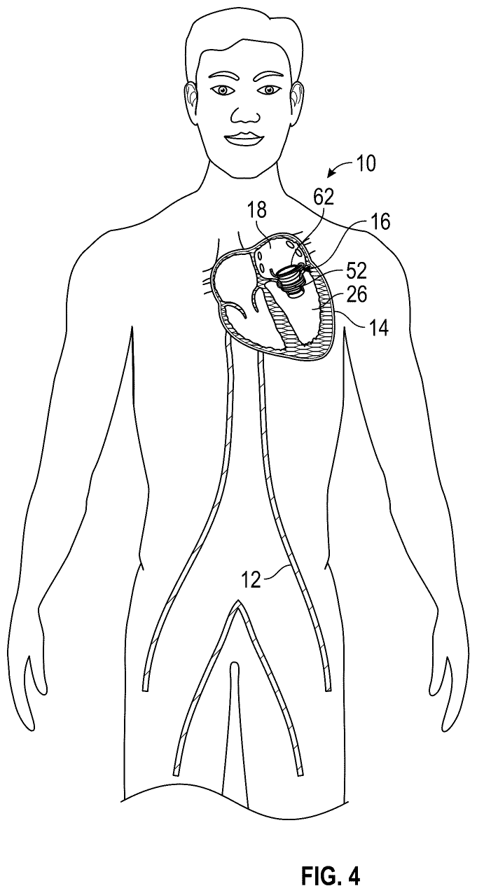

FIGS. 1-4 depict an example of a transcatheter heart valve replacement procedure (such as a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a delivery apparatus 50 (FIG. 2A) and then removes the delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (such as the delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 (which may also be referred to as a catheter shaft 34) extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (such as the guide catheter 30, the delivery apparatus 50, the prosthetic valve delivery apparatus 60, additional catheters, or the like) and their associated devices (such as docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle 26 of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (such as through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a delivery apparatus 50 (which may also be referred to as an “implant catheter,” a dock delivery system,” a “docking device delivery apparatus,” and/or a “docking device delivery device”).

In general, the delivery apparatus 50 comprises a delivery shaft 54 (which may also be referred to as a “dock delivery system shaft”), a handle 56 (which may also be referred to as a “dock delivery system handle”), and a pusher assembly 58. The delivery shaft 54 is configured to be advanced through the patient's vasculature (blood vessel 12) and to the implantation site (such as native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the delivery apparatus 50 is configured to be gripped and/or otherwise held by the user, outside the body of the patient 10, to advance the delivery shaft 54 through the patient's vasculature (such as the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (such as the native mitral valve 16). For example, the pusher assembly 58 is configured to be adjusted by the user to push the docking device 52 out of the distal end portion 53 of the delivery shaft 54. A shaft (which may also be referred to as a “pusher shaft”) of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the delivery apparatus 50 such that the docking device 52 can be released after being deployed at the native mitral valve 16.

Further details of the docking device delivery apparatus and its variants are described in PCT Publication Nos. WO2020/247907 and WO2023/205076 and PCT Application No. PCT/US2023/33745, each of which are incorporated by reference herein in their entirety.

Referring again to FIG. 2A, after the guide catheter 30 is positioned within the left atrium 18, the user may insert the delivery apparatus 50 (such as the delivery shaft 54) into the patient 10 by advancing the delivery shaft 54 of the delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the delivery apparatus 50 through the blood vessel 12 along the guidewire 40 until the delivery shaft 54 reaches the left atrium 18, as illustrated in FIG. 2A. Specifically, the user may advance the delivery shaft 54 of the delivery apparatus 50 by gripping and exerting a force on (for example, by pushing) the handle 56 of the delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (such as the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (such as the portion of the docking device 52 shown in FIG. 2A that is configured to be positioned within the left ventricle 26 and/or on the ventricular side of the native mitral valve 16), the user may then deploy the remaining portion of the docking device 52 (such as an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the delivery apparatus 50 from the docking device 52. Once the docking device 52 is disconnected from the delivery apparatus 50, the user may retract the delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the delivery apparatus 50 (including the delivery shaft 54) has been removed from the patient 10 such that only the guidewire 40 and the guide catheter 30 remain inside the patient 10. In some examples, after removing the delivery apparatus 50, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (such as the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, by pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, by inflating the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (such as tricuspid, pulmonary, and/or aortic valves). Further, the same and/or similar delivery apparatuses (such as the delivery apparatus 50, prosthetic valve delivery apparatus 60, guide catheter 30, and/or guidewire 40), docking devices (such as the docking device 52), replacement heart valves (such as the prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

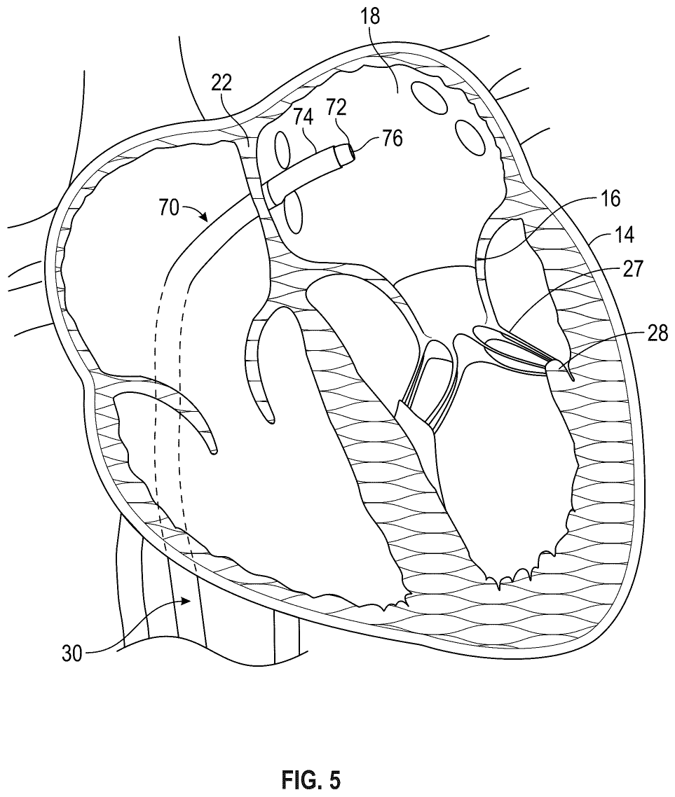

FIGS. 5-11 schematically illustrate a procedure for implanting a prosthetic medical device at a target implantation site in a subject (such as the patient 10). In some examples, the procedure is a docking device implantation procedure for implanting a docking device 152 at an annulus of the native mitral valve 16 of the patient 10. In some examples, one exemplary difference between the docking device 52 of FIGS. 1-4 and the docking device 152 of FIGS. 5-11 can be that the docking device 152 optionally includes a guard member 180 coupled to the docking device 152, wherein the guard member 180 can be configured to further mitigate the possibility of paravalvular leakage between the annulus of the native mitral valve 16 and a prosthetic heart valve (such as the prosthetic heart valve 62) positioned in the docking device 152.

The procedure of FIGS. 5-11 can be performed using a delivery apparatus 150 (which may also referred to as a “docking device delivery apparatus”). In some examples, one exemplary difference between the delivery apparatus 150 and the delivery apparatus 50 of FIGS. 1-4 is that the delivery apparatus 150 can comprise three independently actuatable shafts: a delivery shaft 154 (which may also be referred to as a “dock delivery system shaft”), a sleeve shaft 182, and a pusher shaft 184 (which may also be referred to as a “dock shaft”). The pusher shaft 184 can be disposed within the sleeve shaft 182, which can in turn be disposed within the delivery shaft 154. In some examples, the delivery shaft 154, the sleeve shaft 182, and the pusher shaft 184 can be coaxial. The delivery shaft 154, the sleeve shaft 182, and the pusher shaft 184 can be independently actuated relative to one another in the axial direction during the docking device implantation procedure to better position the docking device 152 within the annulus of native mitral valve 16, such that the implanted docking device 152 can better encircle one or more chordae tendineae 27 of the heart 14 and provide for better sealing between the implantation site and a prosthetic heart valve (such as the prosthetic heart valve 62).

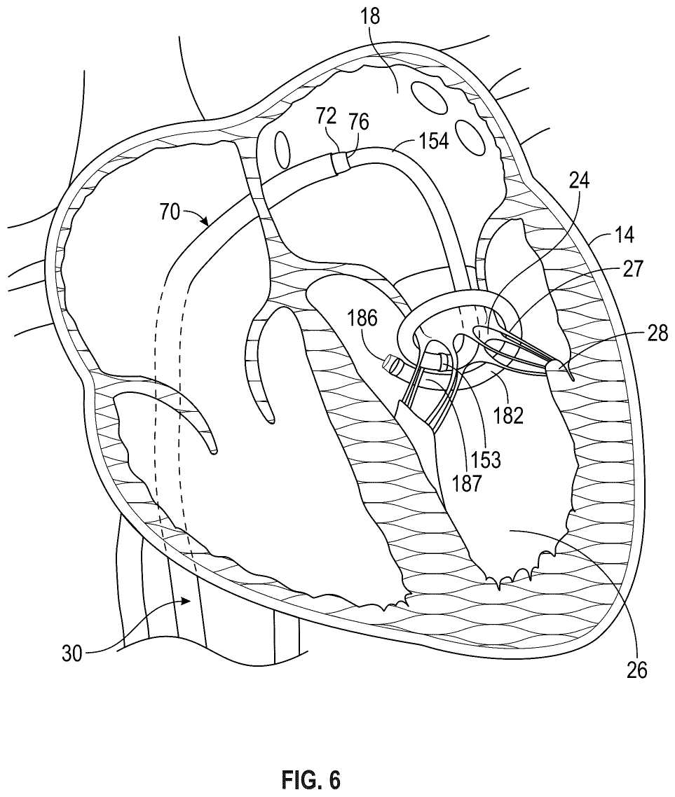

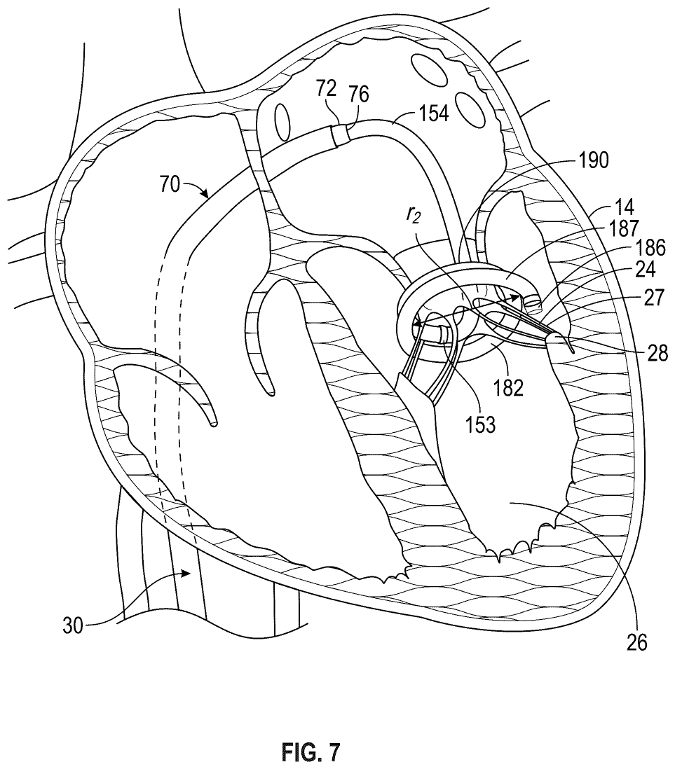

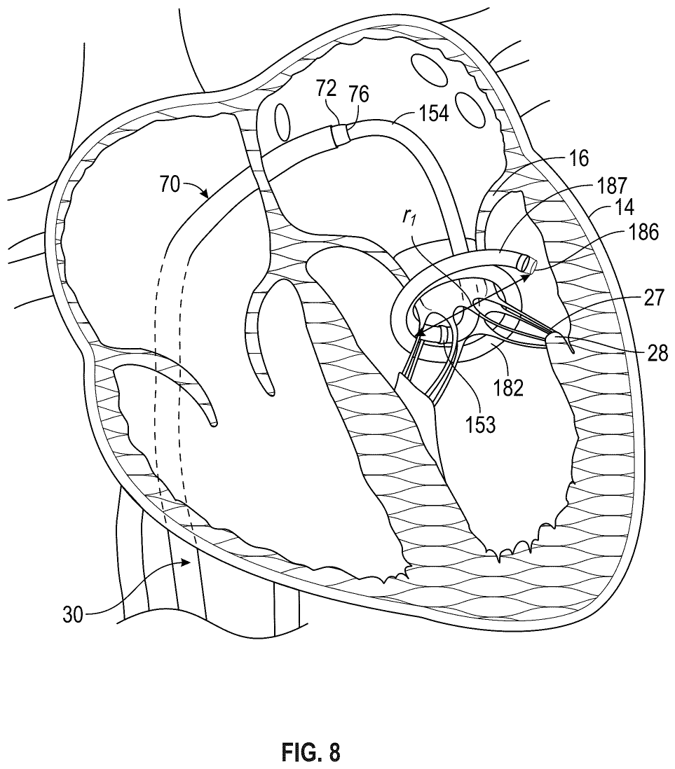

During the procedure, a user of the delivery apparatus 150 first creates a pathway to a patient's native heart valve using the guide catheter 30 (FIG. 5). The user then distally advances a distal end portion of the delivery apparatus 150 to advance the docking device 152 to the target implantation site (FIG. 6-7). In some examples of the procedure, the user can acuate the delivery system to change or adjust the curvature of the distal end portion of the delivery apparatus 150 (see, for example, the leading turn 187 of the delivery apparatus 150 in FIGS. 7-8). This adjustable radius of curvature can be referred to as a “variable encircling turn” (VET). The VET can, for example, make it easier to encircle one or more chordae tendineae 27 connecting the leaflets 24 to the papillary muscles 28 of the heart 14 by proximally retracting the pusher shaft 184 relative to the sleeve shaft 182.

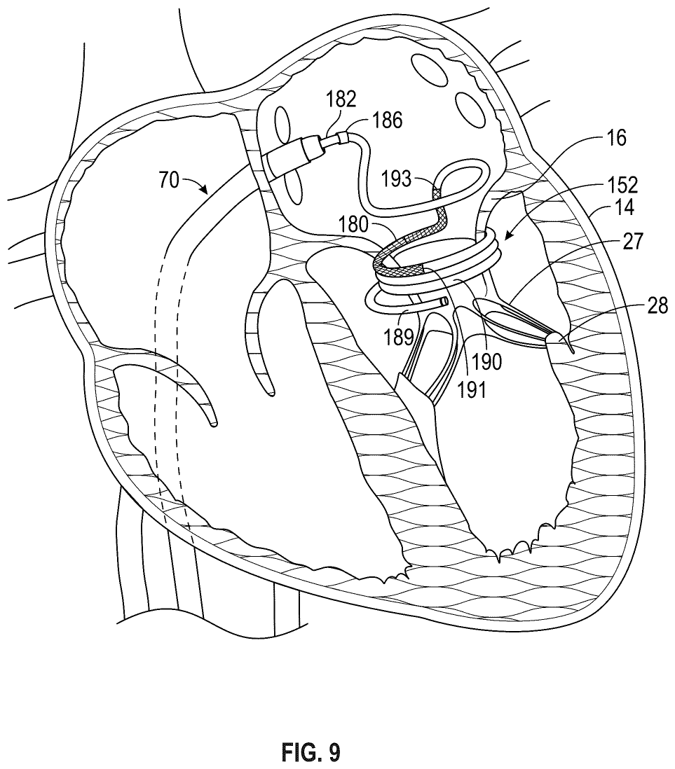

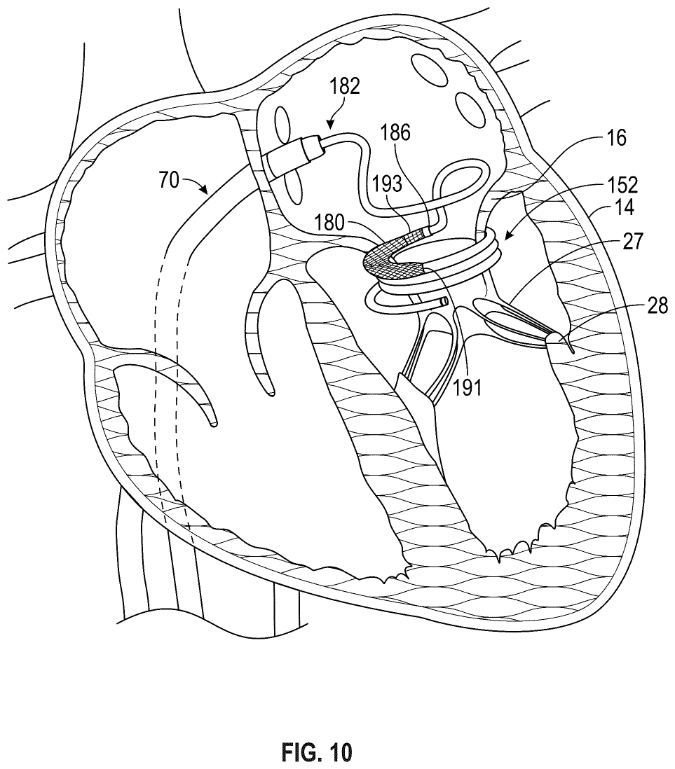

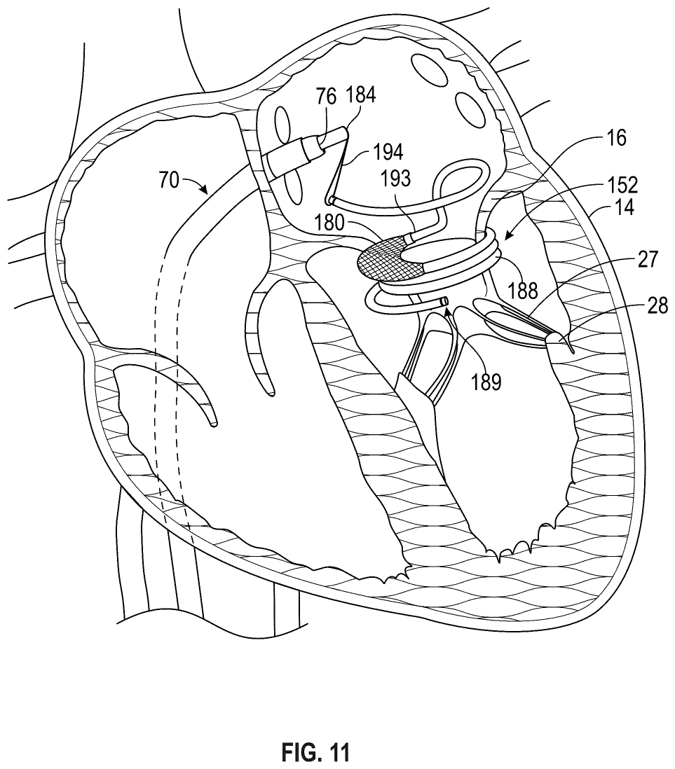

In some examples where the docking device 152 further comprises a guard member 180, the user can then retract the delivery shaft 154 and the sleeve shaft 182 in the proximal direction to expose the guard member 180 (FIG. 9) from the sleeve shaft 182. In some examples, the user can then advance the sleeve shaft 182 in the distal direction to exert an axially compressive force against the guard member 180, thereby axially foreshortening and radially expanding the guard member 180 (FIG. 10). Finally, the user can decouple the docking device 152 from the pusher shaft 184 and remove the delivery device system 150 from the patient 10 (FIG. 11).

FIG. 5 illustrates a stage in the procedure in which the guide catheter 30 is advanced in a distal direction through the patient's vasculature and into the left atrium 18 of the heart 14. The guide catheter 30 comprises the catheter shaft 34 that includes a distal end 72, a flex region 74, and a lumen exit 76 on the distal end 72 of the catheter shaft 34. The lumen exit 76 is connected to a catheter shaft lumen disposed within the catheter shaft 34. A delivery apparatus (such as any of the prosthetic device delivery apparatuses or implant catheters described herein) is configured to be disposed within the catheter shaft lumen. The catheter shaft lumen extends from a proximal end portion of the catheter shaft 34 (such as the portion of the catheter shaft 34 coupled to the handle 32) to the lumen exit 76. The guide catheter 30 is positioned such that the distal end 72 of the catheter shaft 34 is disposed within the left atrium 18 of the heart 14.

In some examples, the catheter shaft 34 can comprise one or more pull wires for adjusting a curvature of the flex region 74 of the catheter shaft 34. In some examples, the pull wires can extend through a lumen coupled to the lumen exit 76 and can couple to a portion of the catheter shaft 34, such as a pull wire ring at or adjacent the distal end 72. In some examples, the pull wires can extend through one or more pull wire lumens embedded in the catheter shaft 34. In some examples, adjusting a tension of the pull wires can adjust the curvature of a flex region 74 of the catheter shaft 34. In some instances, the catheter shaft 34 (including its flex region 74) can be integrally formed as a single, unitary component. In some instances, the catheter shaft 34 can comprise one or more segments (for example, the flex region 74, other regions, etc.) that are formed as separate components that are coupled together (such as via fasteners, adhesive, mating features, and/or other means for coupling). In some examples, the flex region 74 can comprise a material that is more prone to flexing, bending, twisting, etc. than the remaining portion of the catheter shaft 34 (for example, a polymer having relatively lower durometer hardness). This can enable the curvature of the flex region 74 to be adjusted or increased at a different rate than the remaining portion of the catheter shaft 34 when the pull wires are tensioned. For example, the curvature of the flex region 74 can change at an increased rate relative to the proximal portion of the catheter shaft 34 as the tension of the pull wires is increased. The catheter shaft 34 can also include one or more reinforcing braids or jackets that makes the catheter shaft 34 more resistant to flexing, bending, twisting, etc., for example, to prevent one or more of the lumens from kinking or collapsing when the catheter shaft 34 is manipulated.

During this stage, the docking device 152 is disposed within the sleeve shaft 182, which in turn is disposed within the delivery shaft 154, which in turn is disposed within the catheter shaft 34. The pusher shaft 184 is disposed proximally adjacent the docking device 152 within the sleeve shaft 182. In some examples, the docking device 152, the sleeve shaft 172, the delivery shaft 154, and the catheter shaft 34 can be coaxially aligned. During this stage, the docking device 152 is in a generally straight delivery configuration (that is, without any coiled or looped portions, but can be flexed or bent) so as to maintain a small radial profile when moving through a patient's vasculature.

FIG. 6 illustrates a stage in the procedure in which the docking device 152, the delivery shaft 154, the sleeve shaft 182, and the pusher shaft 184 are advanced in a distal direction through the lumen exit 76 of the catheter shaft 34, through the left atrium 18, and to the native mitral valve 16. The docking device 152 is disposed within a sleeve shaft lumen of the sleeve shaft 182, which in turn is disposed within a delivery shaft lumen of the delivery shaft 154. The pusher shaft 184 is disposed proximally adjacent the docking device 152 within the sleeve shaft 182.

The delivery shaft 154, which in some examples can resemble the delivery shaft 54, comprises the delivery shaft lumen through which the sleeve shaft 182 and the pusher shaft 184 can extend. The delivery shaft lumen is configured to extend in the axial direction along the length of the delivery shaft 154 between a handle of the delivery apparatus 150 and a distal end portion 153 of the delivery shaft 154. The sleeve shaft 182 and the pusher shaft 184 are configured to exit the delivery shaft lumen through an opening at the distal end portion 153.

The sleeve shaft 182 is configured to extend through the delivery shaft 154 and sheathe the docking device 152 and at least a portion of the pusher shaft 184 as the docking device 152 is navigated through the patient's vasculature to the native mitral valve 16. The sleeve shaft 182 comprises the sleeve shaft lumen extending along the length of the sleeve shaft 182 between a handle of the delivery apparatus 150 and a distal end portion 186 of the sleeve shaft 182. In some examples, a portion (for example, a proximal end portion) of the sleeve shaft 182 can have a substantially U-shaped axial cross-section or other shape that allows the proximal end portion of the pusher shaft 184 to exit the sleeve shaft 182. The distal end portion of the pusher shaft 184 can exit the sleeve shaft 182 at an opening at the distal end portion 186 of the sleeve shaft 182.

The distal end portion 186 of the sleeve shaft 182 is configured to capture the native tissue (for example, the native leaflets 24 and chordae 27). The sleeve shaft 182 can have a relatively low-friction and/or lubricious outer surface to reduce the likelihood of the sleeve shaft 182 snagging on the native tissue.

In some examples, the sleeve shaft 182 can comprise a plurality of layers. For example, the sleeve shaft 182 can comprise an inner-most polymeric layer, a braided or other type of flexible reinforcing layer, and an outer-most polymeric layer. In some examples, the reinforcing layer is a shape memory material and/or elastic material (for example, nitinol and/or stainless steel).

In some instances, the distal end portion 186 of the sleeve shaft 182 can be curved to help facilitate the capture of the native tissue. This can be accomplished by forming the distal end portion 186 of the sleeve shaft 182 in a curved configuration and/or by forming the sleeve shaft 182 of a relatively more flexible material than the docking device 152 and advancing the curved docking device 152 into the sleeve shaft 182, which can result in the sleeve shaft 182 assuming a curved configuration and/or the curvature of the sleeve shaft 182 being altered by the docking device 152.

In this manner, the distal end portion 186 of the sleeve shaft 182 can form a sleeve shaft leading turn 187 configured to capture the chordae tendineae 27 as the sleeve shaft 182 is advanced around the leaflets 24 of the native mitral valve 16. The sleeve shaft leading turn 187 is a portion of the sleeve shaft 182 disposed at or adjacent the distal end portion 186 that comprises a curved portion of the sleeve shaft 182 having a radius of curvature. When the docking device 152 is not sheathed within the portion of the sleeve shaft 182 corresponding to the sleeve shaft leading turn 187, the sleeve shaft leading turn 187 has a radius of curvature equal to a first radius of curvature (r₁) As discussed later in this application, particularly in reference to FIGS. 7-8, the radius of curvature of the sleeve shaft leading turn 187 can be varied by relative movement between the sleeve shaft 182 and the docking device 152. In some examples, in which the sleeve shaft 182 can be constructed from, formed of, and/or comprise a shape memory material, the sleeve shaft 182 may originally be formed such that the sleeve shaft leading turn 187 has the first radius of curvature (r₁). The sleeve shaft leading turn 187 can be forced into another configuration having another radius of curvature (for example, second radius of curvature (r₂)) but can revert to its original configuration having the first radius of curvature (r₁) when the force is removed. In some examples, the second radius of curvature (r₂) can be less than the first radius of curvature (r₁).

In some examples, the sleeve shaft leading turn 187 can conform to a shape or curvature of another component (such as the docking device 152) sheathed by the sleeve shaft leading turn 187, such that the radius of curvature of the sleeve shaft leading turn 187 is equal to a corresponding radius of curvature of the other component. As such, the distal end portion 186 of the sleeve shaft 182 can have a lesser radius of curvature when a distal end portion (such as leading turn 189) of the docking device 152 is disposed at or proximate to the distal end portion 186 of the sleeve shaft 182. This is because the docking device 152 can have a smaller radius of curvature and can be relatively more rigid than the sleeve shaft 182. In some examples, the radius of curvature of the distal end portion 186 of the sleeve shaft 182 can be increased by moving the distal end of the docking device 152 proximally relative to the distal end portion 186 of the sleeve shaft 182 such that the sleeve shaft 182 can assume its pre-set configuration. This can be done by moving the docking device 152 proximally while maintaining the position of the sleeve shaft 182, by moving the sleeve shaft 182 distally relative to the docking device 152, or a combination of the two.

The pusher shaft 184 is configured to extend through the delivery shaft 154 and the sleeve shaft 182. The pusher shaft 184 is configured to be disposed proximally adjacent the docking device 152 within the sleeve shaft 182 while the docking device 152 is navigated through the patient's vasculature to the native mitral valve 16. When the pusher shaft 184 is moved in the axial direction relative to the sleeve shaft 182, the pusher shaft 184 can exert a force on the docking device 152 to move the docking device 152 in the axial direction. In some examples, the docking device 152 can be releasably coupled to the pusher shaft 184 via a connection mechanism of the delivery apparatus 150 such that the docking device 152 can be released after being deployed at the native mitral valve 16.

In some examples, the distal end portion 153 of the delivery shaft 154 can be positioned during this stage between the leaflets 24 of the native mitral valve 16 (such as at or near the posteromedial commissure). In some examples, the distal end portion 153 of the delivery shaft 154 can extend distally past the native mitral valve 16 and be positioned adjacent the native mitral valve 16 in the left ventricle 26. In some examples, the distal end portion 153 of the delivery shaft 154 can be positioned adjacent the native mitral valve 16 in the left atrium 18.

Once the distal end portion 153 of the delivery shaft 154 is positioned, the docking device 152, the sleeve shaft 182, and the pusher shaft 184 are advanced in a distal direction out of the opening at the distal end portion 153 of the delivery shaft 154, through the native mitral valve 16, and into the left ventricle 26.

FIG. 7 illustrates a stage in the procedure in which the docking device 152 (disposed within the sleeve shaft 182), the sleeve shaft 182, and the pusher shaft 184 (disposed within the sleeve shaft 182 and proximally adjacent the docking device 152) wrap around or encircle the leaflets 24 on the ventricular side of the native mitral valve 16. When the docking device 152 exits the delivery shaft 154, the docking device 152 assumes a coiled configuration that is configured to wrap around or encircle the leaflets 24 on the ventricular side of the native mitral valve 16. In some examples, in which the docking device 152 can be constructed from, formed of, and/or comprise a shape memory material, the docking device 152 may originally be formed in the coiled configuration, but may be forced into a straightened delivery configuration by the delivery shaft 154. The docking device 152 can assume its original coiled configuration once the docking device 152 is no longer sheathed by the delivery shaft 154.

As previously discussed, the portions of the sleeve shaft 182 that sheathe the docking device 152 can conform to or assume the shape and/or curvature of corresponding portions of the docking device 152. For example, the sleeve shaft leading turn 187 can conform to the leading turn 189 of the docking device 152, wherein the leading turn 189 has radius of curvature equal to the second radius of curvature (r₂). Thus, the sleeve shaft leading turn 187 can assume a configuration having the second radius of curvature (r₂). In other words, the variable encircling turn can be equal to the second radius of curvature (r₂).

FIG. 8 illustrates an optional stage in the procedure in which the radius of curvature of the sleeve shaft leading turn 187 (in other words, the variable encircling turn) is increased from the second radius of curvature (r₂) to the first radius of curvature (r₁) to better capture the chordae tendineae 27 within the docking device leading turn 189. In some examples, the radius of curvature of the sleeve shaft leading turn 187 can be increased by retracting the pusher shaft 184 in the proximal direction relative to the sleeve shaft 182, such that docking device leading turn 189 and/or the docking device 152 is no longer sheathed by the sleeve shaft leading turn 187. In some examples, the radius of curvature of the sleeve shaft leading turn 187 can be increased by advancing the distal end portion 186 of the sleeve shaft 182 in the distal direction relative to the docking device 152. When the sleeve shaft leading turn 187 is no longer forced to conform to the curvature of the docking device leading turn 189 having the second radius of curvature (r₂), the sleeve shaft leading turn 187 can revert to its original configuration having the first radius of curvature (r₁), which is larger than the second radius of curvature (r₂). Since the chordae tendineae 27 are captured within the sleeve shaft leading turn 187, increasing the variable encircling turn to the larger first radius of curvature (r₁) beneficially allows for more portions of the chordae tendineae 27 to be captured by the sleeve shaft leading turn 187 as it is advanced around the leaflets 24.

During the step illustrated in FIG. 8, the delivery shaft 154 can be kept stationary to preserve the position of the distal end portion 153 of the delivery shaft 154 relative to the native mitral valve 16 (such as at or near the posteromedial commissure). In some examples, the sleeve shaft 182 can be kept stationary to preserve the encircling position and/or radial orientation of the sleeve shaft 182 relative to the native mitral valve 16. In some examples, the docking device 152 and/or the pusher shaft 184 can be kept stationary while the sleeve shaft 182 is moved during this step. In some examples, neither the sleeve shaft nor the pusher shaft 184 are kept stationary during this step.

As shown in FIG. 8, the variable encircling turn can be adjusted after the sleeve shaft 182 has made one helical turn around the leaflets 24. However, in some examples, the variable encircling turn can be adjusted after the sleeve shaft 182 has formed a plurality of helical turns around the leaflets 24. In some examples, the variable encircling turn can be adjusted before any helical turns have been formed around the leaflets 24.

FIG. 9 illustrates an optional “unsheathing” stage in the procedure in which the delivery shaft 154 and the sleeve shaft 182 are retracted in the proximal direction to unsheathe the guard member 180. The docking device 152 comprises a coil 188 that defines a central region 190 comprising a plurality of helical turns wrapped around the leaflets 24 and a docking device leading turn 189 extending from a distal end portion of the central region 190.

The docking device 152 can further comprise the guard member 180 disposed on the docking device 152 such that the guard member 180 is positioned at or near the native mitral valve 16 (such as at or near the posteromedial commissure) when the docking device 152 is implanted at the native mitral valve 16. In some examples, the guard member 180 can be disposed proximally adjacent a central region (FIG. 22), wherein the central region can comprise a plurality of helical turns when the docking device 152 is wrapped around the leaflets 24. The guard member 180 can extend between a distal end portion 191 that is fixedly coupled to the docking device 152 and a movable proximal end portion 193 that can be moved along at least a portion of the docking device 152 in the axial direction. In some examples, the distal end portion 191 of the guard member 180 can abut the central region 190.

During the stages of the docking device implantation procedure shown in FIGS. 5-8, the guard member 180 can be covered by the delivery shaft 154 and the sleeve shaft 182. However, during the stage illustrated in FIG. 9, relative movement between the delivery shaft 154, the sleeve shaft 182, and the pusher shaft 184 can unsheathe the guard member 180. In some examples, the sleeve shaft 182 can be retracted in the proximal direction from the left ventricle 26, through the mitral valve 16, and into the left atrium 18 such that the distal end portion 186 of the sleeve shaft 182 is proximally closer to the user than the proximal end portion 193 of the guard member 180. In some examples, the distal end portion 186 of the sleeve shaft 182 can be distally disposed relative to the lumen exit 76. In some examples, the guard member 180 can be unsheathed by advancing the pusher shaft 184 distally relative to the sleeve shaft 182.

In some examples, the delivery shaft 154 can be retracted through the left atrium 18 in the proximal direction such that the distal end portion 153 of the delivery shaft 154 is proximally closer to the user than the proximal end portion 193 of the guard member 180. In some examples, the delivery shaft 154 can be retracted through the lumen exit 76 and into the catheter shaft lumen of the catheter shaft 34. In some examples, the pusher shaft 184 can be advanced distally relative to the delivery shaft 154 such that the distal end portion 153 of the delivery shaft 154 is proximally disposed relative to the guard member 180.

FIG. 10 illustrates an optional “seating” stage in the procedure in which the sleeve shaft 182 is distally advanced relative to the docking device 152 to axially foreshorten and radially expand the guard member 180. In some examples, the sleeve shaft 182 can be advanced in the distal direction such that the distal end portion 186 of the sleeve shaft 182 abuts and contacts the proximal end portion 193 of the guard member 180. In some examples, the pusher shaft 184 (and the docking device 152 coupled to the pusher shaft 184) can be retracted in the proximal direction such that the distal end portion 186 of the sleeve shaft 182 abuts and contacts the proximal end portion 193 of the guard member 180. The sleeve shaft 182 exerts a force upon the guard member 180 to distally advance the proximal end portion 193 of the guard member 180 relative to the docking device 152. Since the distal end portion 191 of the guard member 180 is fixedly coupled to the docking device 152, exerting the force upon the guard member 180 axially foreshortens and radially expands the guard member 180 to a deployed configuration. When in the deployed configuration, the guard member 180 further reduces the possibility of paravalvular leakage between the native mitral valve 16 and a prosthetic heart valve (such as the prosthetic heart valve 62). The frictional engagement between the proximal end of the guard member 180 and the docking device 152 can retain the position of the guard member 180 relative to the docking device 152 when the sleeve shaft 182 is retracted from the proximal end of the guard member 180. In some examples, the method can further comprise an optional “atrial reveal” stage between the optional unsheathing and seating stages in which the handle 156 is retracted in the proximal direction relative to the sleeve shaft 182.

FIG. 11 illustrates a stage in the procedure in which the delivery apparatus 150, including the delivery shaft 154 and the sleeve shaft 182, are retracted through the catheter shaft lumen of the catheter shaft 34. In some examples, the docking device 152 can be connected to the pusher shaft 184 via a release suture 194 that can be configured to be tied to the docking device 152. The release suture 194 can be cut during this stage to release the docking device 152 from the delivery apparatus 150.

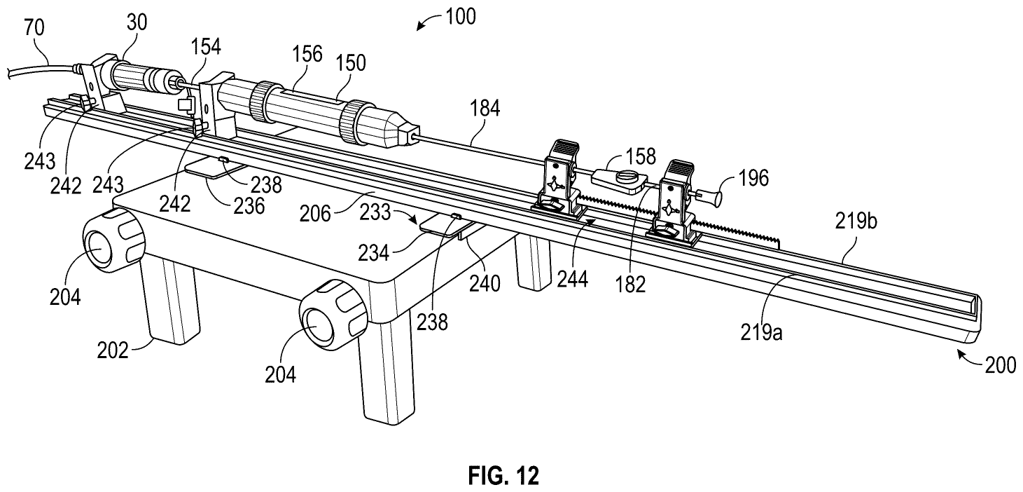

FIG. 12 illustrates an example delivery assembly 100 (which can also be referred to as a “docking device delivery assembly,” a “docking device delivery system,” or a “delivery system”) that can be used in the procedure for implanting a prosthetic medical device, as described above with reference to FIGS. 5-11. The delivery assembly 100 includes the delivery apparatus 150, the guide catheter 30, and a stabilizer assembly 200 (which may also be referred to as a “stabilizing device”) configured to stabilize the delivery apparatus 150 and/or the guide catheter 30 during the procedure.

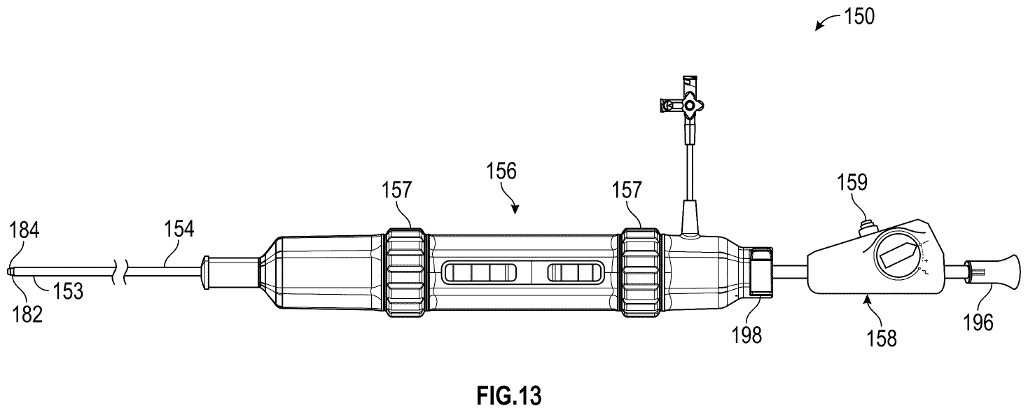

FIG. 13 illustrates the delivery apparatus 150, according to one example. The delivery apparatus 150 can also be referred to as a “dock delivery apparatus,” “dock delivery catheter,” or “dock delivery system.” The delivery apparatus 150 comprises the delivery shaft 154, a handle 156 (which may also be referred to as a “dock delivery system handle” or a “dock handle”) coupled to a proximal end portion of the delivery shaft 154, a sleeve shaft 182 configured to extend through the delivery shaft 154 and the handle 156, a hub assembly 158 (which may also be referred to as a “dock handle”) coupled to a proximal end portion of the sleeve shaft 182, the pusher shaft 184 configured to extend through the handle 156 and the sleeve shaft 182, and a sleeve handle 196 coupled to a proximal end portion of the sleeve shaft 182.

The delivery shaft 154, which in some examples can be similar to the delivery shaft 54, is configured to be advanced through the patient's vasculature (blood vessel 12) and to the implantation site (such as native mitral valve 16) by the user and may be configured to retain the docking device 152 in a distal end portion 153 of the delivery shaft 154. During the docking device implantation procedure, the delivery shaft 154 is advanced through the catheter shaft 34 of the guide catheter 30 (for example, through a central lumen thereof, etc.) and to the target implantation site.

The handle 156, which in some examples can be similar to the handle 56, is configured to be gripped and/or otherwise held by the user, outside the body of the patient 10, to advance the delivery shaft 154 through the patient's vasculature (such as the blood vessel 12). In some examples, the handle 156 can comprise one or more articulation members 157 (such as rotatable knobs) that are configured to aid in navigating the delivery shaft 154 through the blood vessel 12 by steering or controlling the flexing of the delivery apparatus 150 (for example, the delivery shaft 154, etc.). Some examples of the articulation members 157 can be similar to the articulation members 57. The handle 156 comprises a handle lumen extending through the length of the handle 156, wherein the sleeve shaft 182 and the pusher shaft 184 are configured to be disposed within the handle lumen. Since the sleeve and pusher shafts 182, 184 extending through the handle lumen also extend through the delivery shaft 154, the handle lumen can be coaxially aligned with the delivery shaft 154. In some examples, the handle 156 can further comprise a locking assembly 198 configured to lock a device (for example, the sleeve shaft 182) inserted through the handle lumen, such that the device is selectively prevented from moving relative to the handle 156 of the delivery apparatus 150. In some examples, the locking assembly 198 can be disposed on a proximal end portion of the handle 156.

The hub assembly 158 is configured to be gripped and/or otherwise held by the user, outside the body of the patient 10, to advance the pusher shaft 184 through the patient's vasculature. A distal end portion of the hub assembly 158 is coupled to a proximal end portion of the pusher shaft 184. The axial position of the pusher shaft 184 is controlled by moving the hub assembly 158 in the axial direction relative to the handle 156 and/or the sleeve handle 196. The hub assembly 158 is disposed proximally closer to the user relative to the handle 156, but distally further from the user relative to the sleeve handle 196. The hub assembly 158 comprises a hub assembly lumen extending through the length of the hub assembly 158. The pusher shaft 184 is configured to be disposed within the hub assembly lumen and is coaxial with the sleeve shaft 182 coupled to the distal end portion of the hub assembly 158. In some examples, the hub assembly 158 further comprises a suture lock assembly 159 configured to releasably couple to a proximal end of the release suture 194.

The sleeve handle 196 is configured to be gripped and/or otherwise held by the user, outside the body of the patient 10, to advance the sleeve shaft 182 through the patient's vasculature. The sleeve handle 196 is coupled to a proximal end portion of the sleeve shaft 182 and is disposed proximally closer to the user relative to the handle 156 and the hub assembly 158. The axial position of the sleeve shaft 182 is controlled by moving the sleeve handle 196 in the axial direction relative to the handle 156 and/or the hub assembly 158.

Further details on delivery apparatus/catheters/systems (including various examples of the handle assembly) that are configured to deliver a docking device to a target implantation site can be found in PCT Publication Nos. WO 2020/247907 and WO 2022/072509, and U.S. Patent Publication Nos. 2018/0318079 and 2018/0263764, which are all incorporated by reference herein in their entireties.

Since the variable encircling turn can be adjusted based on relative movement between the pusher shaft 184 and the sleeve shaft 182, the user of the docking device apparatus 150 can adjust the radius of curvature of the variable encircling turn (as shown in FIGS. 7-8) by moving the pusher shaft 184 in the axial direction relative to the sleeve shaft 182, or vice versa. Since the pusher shaft 184 is coupled to the hub assembly 158 and the sleeve shaft is coupled to the sleeve handle 196, the variable encircling turn can be adjusted in some examples by moving the hub assembly 158 in the distal direction relative to the sleeve handle 196 while the sleeve handle 196 is kept stationary. In some examples, the sleeve handle 196 can be moved in the proximal direction while the hub assembly 158 is kept stationary. In some examples, both the sleeve handle 196 and the hub assembly 158 can be moved in the axial direction. In some examples, the handle 156 can be kept stationary or can be moved relative to at least one of the sleeve handle 196 and the hub assembly 158.

Referring back to FIG. 12, the guide catheter 30 and the delivery apparatus 150 are configured to be coupled to the stabilizer assembly 200, which, among other things, can support and stabilize the guide catheter 30 and the delivery apparatus 150 during the procedure. The stabilizer assembly 200 includes a platform 202, a stabilizer track 206 mounted to the platform 202, one or more supports 242 (for example, clips, clamps, braces, etc.) that can be slidably coupled to the stabilizer track 206, and a hub assembly support 244 that can be slidably coupled to the stabilizer track 206.

The platform 202 is a table configured to support the stabilizer track 206. The platform 202 is configured to have an adjustable height and/or orientation, wherein the height and/or orientation can be adjusted relative to a surface on which the platform 202 rests (for example, a ground surface or an additional table surface). In some examples, the platform 202 can comprise one or more articulation members 204 (for example, rotatable knobs) for adjusting the height or orientation of the platform 202.

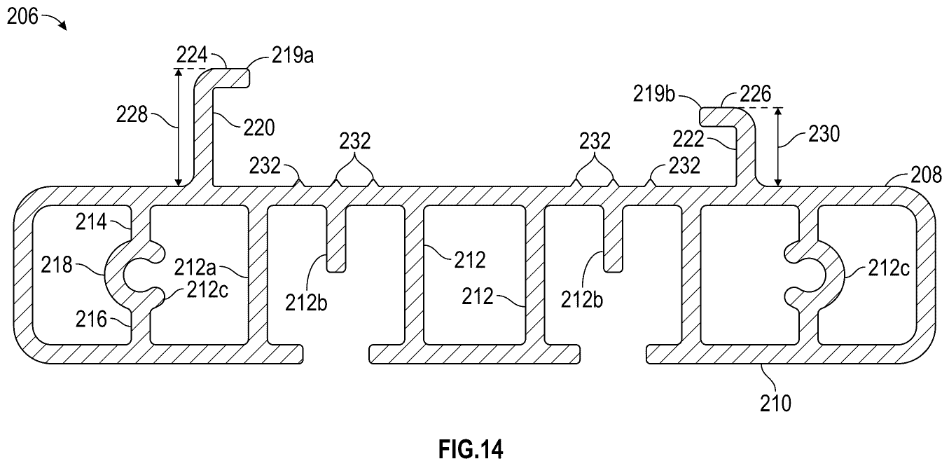

FIG. 14 is an axial cross-sectional view of the stabilizer track 206, according to one example. The stabilizer track 206 comprises a top portion 208 (which is also referred to herein as a “top plate”) extending in the axial direction, a bottom portion 210 (which is also referred to herein as a “bottom plate”) extending in the axial direction, and at least one stiffening beam 212 (which is also referred to herein as a “stiffening plate,” a “stiffening web,” or a ‘stiffening brace”) extending in the axial direction. The stiffening beam 212 can, among other things, increase the stiffness of the stabilizer track 206 and reduce vertical deflections along the length of the stabilizer track 206. The stiffening beam 212 extends along an axial length of the stabilizer track 206. In some examples, the stiffening beam 212 can extend along the entire axial length of the stabilizer track 206. In some examples, the stiffening beam 212 can extend from the top portion 208 towards the bottom portion 210. In some examples, the stiffening beam 212 can extend from the bottom portion 210 towards the top portion 208. In some examples, the stiffening beam 212 can be perpendicular to the top portion 208 and/or the bottom portion 210. In some examples, the stiffening beam 212 can extend from the top portion 208 to the bottom portion 210. In some examples, the stiffening beam 212 can extend from the top portion 208 and only partway towards the bottom portion 210. In some examples, in which the stabilizer track 206 comprises a plurality of stiffening beams 212, a first stiffening beam 212a can extend from the top portion 208 to the bottom portion 210 and a second stiffening beam 212b can extend from the top portion 208 and only partway towards the bottom portion 210.

In some examples, the at least one stiffening beam 212c can comprise a top end portion 214 coupled to the top portion 208, a bottom end portion 216 coupled to the bottom portion 210, and an intermediate portion 218 between the top end portion 214 and the bottom end portion 216. In some examples, the intermediate portion 218 can comprise an axial cross-section with an annular shape (such as an O-shape) or semi-annular shape (such as a C-shape) to increase the second moment of area of the stiffening beam 212c, thereby making the stiffening beam 212c more resistant to bending in the axial direction. In some examples, in which the stabilizer track 206 comprises a plurality of stiffening beams, at least one stiffening beam 212c can comprise an intermediate portion 218 with the annular or semi-annular cross-section.

The stabilizer track 206 comprises a first rail 219a comprising a first web 220 and a first head 224 and a second rail 219b comprising a second web 222 and a second head 226. Although the illustrated stabilizer track 206 comprises two rails, the stabilizer track 206 can comprise any suitable number of rails (for example, one rail, three rails, four rails, five rails, etc.). The first web 220 and the second web 222 each comprise a vertically oriented flange that extends from the top portion 208 and along an axial length of the stabilizer track 206. In some examples, at least one of the first web 220 and the second web 222 can extend along the entire axial length of the stabilizer track 206. The first head 224 comprises a laterally oriented flange that extends laterally from a top end portion of the first web 220. In some examples, the first head 224 can extend from the first web 220 in a laterally inward direction toward the second head 226. The second head 226 comprises a laterally oriented flange that extends laterally from a top end portion of the second web 222. In some examples, the second head 226 can extend from the second web 222 in the laterally inward direction toward the first head 224. In some examples, the first web 220 can comprise a first height 228 in the vertical direction and the second web 222 can comprise a second height 230 in the vertical direction. In some examples, the first height 228 can be greater than the second height 230. In some examples, the first height 228 can be equal to the second height 230. In some examples, the first height 228 can be less than the second height 230.

In some examples, the stabilizer track 206 can further comprise at least one ridge 232 extending from the top portion 208 along an axial length of the stabilizer track 206. The at least one ridge 232 is configured to improve frictional engagement between the brake pad of the stabilizer track lock 257 and the stabilizer track 206.

In some examples, the stabilizer track 206 can be formed from a polymer (such as acrylonitrile butadiene styrene (“ABS”)), a metal (such as aluminum), and/or any other suitable material. In some examples, it can be preferable to form the stabilizer track 206 from a low-density or lightweight material to reduce the weight of the stabilizer track 206. In some examples, the entire stabilizer track 206 (including the top surface 208, the bottom surface 210, the first web 220, the second web 222, the first head 224, the second head 226, and the stiffening beam(s) 212) can be extruded as a unitary structure. Extruding the stabilizer track 206 as a unitary structure can improve the manufacturability of the delivery system by beneficially reducing the number of additional components, such as fasteners, needed to assemble the stabilizer track 206.

In some examples, the stabilizer track 206 can further comprise one or more end caps configured be coupled to the ends of the stabilizer track 206. In some examples, the end caps can be press-fit on the ends of the stabilizer track 206.

Referring back to FIG. 12, the stabilizer assembly 200 optionally comprises a vise 233 (which is also referred to herein as a “clamp”) that can, among other things, secure the stabilizer track 206 to the platform 202. The vise 233 comprises a first base plate 234 and a second base plate 236. Each of the first base plate 236 and the second base plate 236 comprises a cradle 238 extending from a top portion of the respective base plate 234, 236, wherein the cradle 238 is configured to receive a portion of the stabilizer track 206. In some examples, the vise 233 can comprise mechanical fasteners to couple the first base plate 234 and the second base plate 236 to the stabilizer track 206. Each of the first base plate 234 and the second base plate 236 comprises a jaw 240 extending from a bottom portion of the respective base plate 234, 236. When the first base plate 234 and the second base plate 236 are coupled to the stabilizer track 206, the jaws 240 of the first base plate 234 and the second base plate 236 form a vise that clamps the platform 202 between the jaws 240, thereby securing the stabilizer track 206 to the platform 204. In some examples, the jaws 240 can be aligned in the axial direction. In some examples, the vise 233 can further comprise a biasing member disposed on at least one of the first base plate 234 and the second base plate 236 to bias the jaws 240 into engagement with the platform 202.

The supports 242 can, among other things, hold or grip the guide catheter 30 and the handle 156 of the delivery apparatus 150. Each of the supports 242 comprises a post configured to be relocated or repositioned on the stabilizer track 206 in an axial direction, wherein the post is configured to couple to a portion (such as a distal portion) of the guide catheter 30 or the handle 156. In some examples, at least one of the supports 242 can include a position lock 243 configured to prevent the support 242 from moving in an axial direction along the stabilizer track 206. In some examples, the position lock 243 can comprise a threaded shaft that is movable between a locked configuration and an unlocked configuration. When the position lock 243 is in the locked configuration, the threaded shaft comes into frictional contact with the stabilizer track 206, wherein the frictional contact prevents the support 242 from moving in the axial direction relative to the stabilizer track 206.

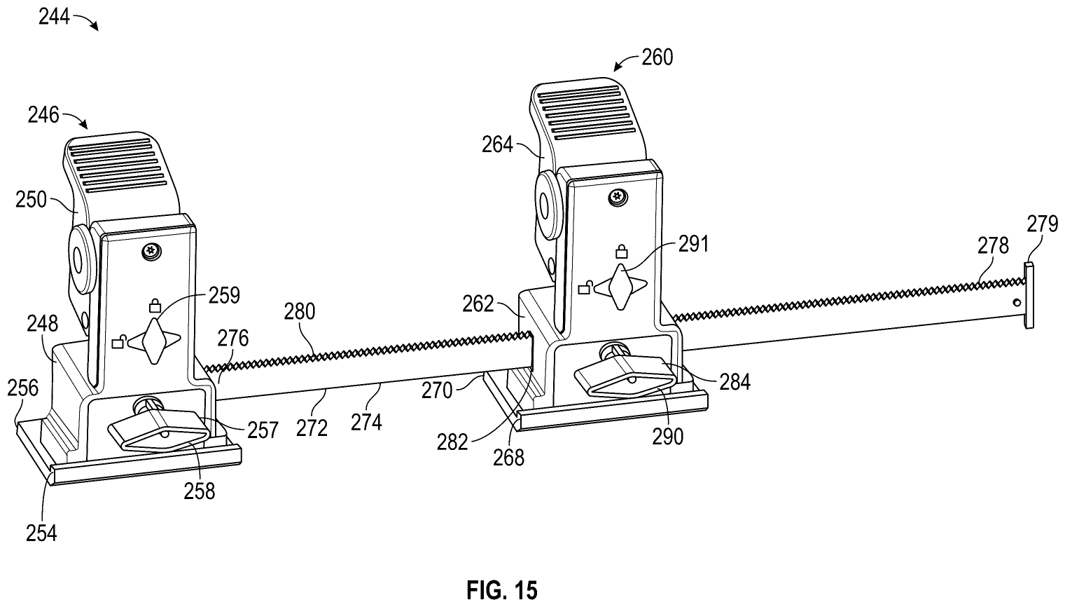

FIG. 15 is a perspective view of the hub assembly support 244 (which is also referred to herein as a “hub assembly support assembly,” a “hub assembly stabilizer,” or a “hub assembly stabilizer assembly”), according to one example. The hub assembly support 244 can, among other things, receive the hub assembly 158 and the sleeve handle 196. In some examples, the hub assembly support 244 can be configured to support, stabilize, and/or align the hub assembly 158 and the sleeve handle 196, thereby supporting, stabilizing, and/or aligning the sleeve shaft 182 and the pusher shaft 184, during the docking device implantation procedure. The hub assembly support 244 comprises a pusher cart 246 coupled to the stabilizer track 206, a sleeve cart 260 coupled to the stabilizer track 206, and a rack 272 extending between the pusher cart 246 and the sleeve cart 260.

The pusher cart 246 (which is also referred to herein as a “pusher post,” a “pusher shaft support,” or a “dock cart”) can, among other things, couple a first portion of the delivery apparatus 150 to the stabilizer track 206. In some examples, the first portion of the delivery apparatus 150 can be a pusher shaft (such as pusher shaft 184) or a hub assembly (such as hub assembly 158) coupled to a proximal end of the pusher shaft. The pusher cart 246 comprises a pusher cart chassis 248 (which is also referred to herein as a “column” or a “post”), a clamp 250 extending from the pusher cart chassis 248, and first and second rail engagement flanges 254, 256 disposed on a bottom portion of the pusher cart chassis 248. The clamp 250 is configured to receive the first portion (such as the pusher shaft 184) of the delivery apparatus 150 (see, for example, FIG. 12) such that the first portion of the delivery apparatus 150 becomes releasably coupled to the pusher cart 246. In some examples, the clamp 250 can extend from a top portion of the pusher cart chassis 248. The first and second rail engagement flanges 254, 256 are configured to couple the pusher cart 246 to the stabilizer track 206. The first rail engagement flange 254 extends in a first lateral direction from the pusher cart chassis 248 and the second rail engagement flange 256 extends in a second lateral direction—which is opposite the first lateral direction—from the pusher cart chassis 248. The first rail engagement flange 254 is configured to nestle under the first head 224 and laterally adjacent the first web 220 of the stabilizer track 206, thereby engaging the first rail 219a of the stabilizer track 206. The second rail engagement flange 256 is configured to nestle under the second head 226 and laterally adjacent the second web 222 of the stabilizer track 206, thereby engaging the second rail 219b of the stabilizer track 206. In some examples where the height 228 of the first web 220 is greater than the height 230 of the second web 222, the first rail engagement flange 254 can have a vertical thickness greater than a vertical thickness of the second rail engagement flange 256.

In some examples, a rail engagement flange (such as the first rail engagement flange 254 or the second rail engagement flange 256) can be actuatable in the lateral direction to facilitate the coupling and/or decoupling of the pusher cart 246 to the stabilizer track 206. In some examples, the rail engagement flange can be slidingly coupled to the pusher cart chassis 248, such that the rail engagement flange can move in the lateral direction between a locked configuration and an unlocked configuration. In the locked configuration, the rail engagement flange extends laterally outward from the pusher cart chassis 248 to nestle under a head (such as the first head 224 or the second head 226) and adjacent a web (such as the first web 220 or the second web 222) of a rail, thereby engaging the rail and locking the pusher cart 246 to the stabilizer track 206. In the unlocked configuration, the rail engagement flange retracts at least partially into the pusher cart chassis 248 to disengage the rail, thereby allowing the pusher cart 246 to be decoupled from the stabilizer track 206. In some examples, the rail engagement flange can be biased in the locked configuration by at least one biasing member. In some examples, the biasing member can be a spring. In some examples, the pusher cart 246 can further comprise a button coupled to the lateral flange. When pressed, the button can actuate the rail engagement flange between the locked configuration and the unlocked configuration.

In some examples, the pusher cart 246 can further comprise a stabilizer track lock 257 (which is also referred to herein as a “brake”) configured to lock the pusher cart 246 to the stabilizer track 206. The stabilizer track lock 257 comprises a brake pad, a cam, and a rotatable knob 258 actuatable between a locked configuration and an unlocked configuration. In the locked configuration, the brake pad extends from the pusher cart chassis 248 and frictionally engages the stabilizer track 206. In some examples, the brake pad can extend from the bottom portion of the pusher cart chassis 248. The brake pad is coupled to the knob 258 via the cam. When the knob 258 is rotated (for example, to a vertical direction), the rotational motion of the knob 258 is translated by the cam into a linear motion (for example, in the vertical direction) to move the brake pad into frictional contact with a portion of the stabilizer track 206 (for example, the top portion 208 of the stabilizer track 206). In some examples, the pusher cart 246 can further comprise a marking 259 that indicates whether the pusher cart 246 is locked to the stabilizer track 206 (in other words, whether the stabilizer track lock 257 is engaged). In some examples, the brake pad can be formed from silicone. In some examples, the brake pad can be formed from any material with a coefficient of friction sufficient to prevent movement of the pusher cart 246 along the stabilizer track 206.

The sleeve cart 260 (which is also referred to herein as a “sleeve post,” or a “sleeve shaft support,”) can, among other things, couple a second portion of the delivery apparatus 150 to the stabilizer track 206. In some examples, the second portion of the delivery apparatus 150 can be a sleeve shaft (such as sleeve shaft 182). In some examples, the second portion of the delivery apparatus 150 can be a sleeve handle (such as sleeve handle 196) coupled to the sleeve shaft. In some examples, the sleeve cart 260 can be proximally disposed on the stabilizer track 206 relative to the pusher cart 246. The sleeve cart 260 comprises a sleeve cart chassis 262, a clamp 264 extending from a top portion of the sleeve cart chassis 262, and first and second rail engagement flanges 268, 270 disposed on a bottom portion of the sleeve cart chassis 262. The clamp 264 is configured to receive the second portion of the delivery apparatus 150. In some examples, the clamp 264 can extend from a top portion of the sleeve cart chassis 262. However, the clamp 264 can extend from any suitable portion of the sleeve cart 260. The first rail engagement flange 268 and the second rail engagement flange 270 are configured to couple the sleeve cart 260 to the stabilizer track 206. The first rail engagement flange 268 extends in a first lateral direction from the sleeve cart chassis 262 and the second rail engagement flange 270 extends in a second lateral direction from the sleeve cart chassis 262, wherein the first lateral direction is opposite the second lateral direction. The first rail engagement flange 268 is configured to nestle under the first head 224 and laterally adjacent the first web 220 of the stabilizer track 206, thereby engaging the first rail 219a of the stabilizer track 206. The second rail engagement flange 270 is configured to nestle under the second head 226 and laterally adjacent the second web 222 of the stabilizer track 206, thereby engaging the second rail 219b of the stabilizer track 206. In some examples where the height 228 of the first web 220 is greater than the height 230 of the second web 222, the first rail engagement flange 268 can have a vertical thickness greater than a vertical thickness of the second rail engagement flange 270.

In some examples, a rail engagement flange (such as the first rail engagement flange 268 or the second rail engagement flange 270) can be actuatable in the lateral direction to facilitate the coupling and/or decoupling of the sleeve cart 260 to the stabilizer track 206. In some examples, the rail engagement flange can be slidingly coupled to the sleeve cart chassis 262, such that the rail engagement flange can move in the lateral direction between a locked configuration and an unlocked configuration. In the locked configuration, the rail engagement flange extends laterally outward from the sleeve cart chassis 262 to nestle under a head (such as the first head 224 or the second head 226) and adjacent a web (such as the first web 220 or the second web 222) of a rail, thereby locking the sleeve cart 260 to the rail of the stabilizer track 206. In the unlocked configuration, the rail engagement flange retracts at least partially into the sleeve cart chassis 262 to disengage the head, thereby allowing the sleeve cart 260 to be decoupled from the rail of the stabilizer track 206. In some examples, the rail engagement flange can be biased in the locked configuration by at least one biasing member. In some examples, the biasing member can be a spring. In some examples, the sleeve cart 260 can further comprise a button coupled to the lateral flange. When pressed, the button can actuate the rail engagement flange between the locked configuration and the unlocked configuration.

The rack 272 (which is also referred to herein as a “rack gear,” a “guide member,” a “guide rack,” a “guide rail,” or a “linear gear”) can, among other things, help regulate the axial distance between the pusher cart 246 and the sleeve cart 260. The rack 272 comprises an axially elongated beam 274 extending in the axial direction that includes a first axial end portion 276 (such as a distal end portion) and a second axial end portion 278 (such as a proximal end portion). In some examples, the first axial end portion 276 can be fixedly coupled to the pusher cart 246. In some examples, the sleeve cart 260 can be configured to slidingly couple to the rack 272, such that the sleeve cart 260 can selectively slide along either a length of the rack 272 and a length of the stabilizer track 206. In some examples, the sleeve cart 260 can slide along the length of the rack 272 between the first axial end portion 276 and the second axial end portion 278. In some examples, the rack 272 can comprise a stopper 279 coupled to the second axial end portion 278 to prevent the sleeve cart 260 from sliding off the rack 272 and past the second axial end portion 278. The rack 272 comprises a plurality of teeth 280 arranged along the beam 274 in the axial direction. In some examples, the sleeve cart 260 can comprise a slot 282 extending through the sleeve cart chassis 262 in the axial direction. The slot 282 can be configured to receive a portion of the rack 272 therein.

In some examples, the hub assembly support 244 can comprise a rod extending between the pusher cart 246 and the sleeve cart 260 instead of the rack 272. One exemplary difference between the rod and the rack 272 is that the rod does not comprise the plurality of teeth 280.

In some examples, the rack 272 can be replaced with a linear actuator configured to regulate the axial separation of the pusher cart 246 and the sleeve cart 260. The linear actuator can comprise a first axial end portion (such as a distal end portion) coupled to the pusher cart 246 and a second axial end portion (such as a proximal end portion) coupled to the sleeve cart 260. The linear actuator can be configured to axially elongate or contract. When the linear actuator elongates or retracts, the linear actuator can move the pusher cart 246 relative to the sleeve cart 260, the sleeve cart 260 relative to the pusher cart 246, or both the pusher cart 246 and the sleeve cart 260 relative to each other. In some examples, the linear actuator can comprise a piston (such as a hydraulic, pneumatic, or piezoelectric piston), a telescopic cylinder, a screw, a pulley, or any other suitable linkage capable of linear motion.

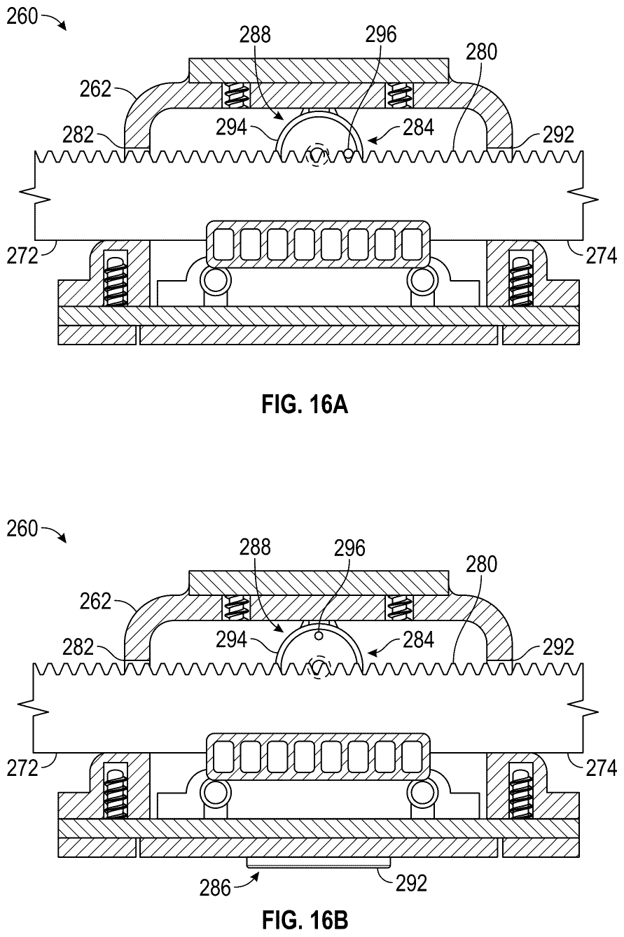

FIGS. 16A-16B show a cross-sectional view of the sleeve cart 260 (and more specifically, a cross-sectional view of the sleeve cart chassis 262), according to one example. The sleeve cart 260 comprises a locking assembly 284 configured to selectively engage (or lock) the stabilizer track 206 and the rack 272. The locking assembly 284 comprises a stabilizer track lock 286 configured to frictionally engage the stabilizer track 206, a rack lock 288 configured to engage the rack 272, and a rotatable knob 290 configured to actuate both the stabilizer track lock 286 and the rack lock 288.

The locking assembly 284 is actuatable between a first mode (which is also referred to herein as a “VET OFF mode”) and a second mode (which is also referred to as a “variable encircling turn mode,” “VET mode,” or “VET ON mode”).

When the locking assembly 284 is in the first mode (FIG. 16A), the stabilizer track lock 286 (FIG. 16B) disengages the stabilizer track 206 and the rack lock 288 engages the teeth 280 of the rack 272, thereby maintaining a constant axial separation distance between the pusher cart 246 and the sleeve cart 260. Since the locking assembly 284 prevents relative movement between the pusher cart 246 and the sleeve cart 260 (and thus relative movement between the pusher shaft 184 and the sleeve shaft 182), thereby allowing the pusher cart 246 and the sleeve cart 260 to be moved along the stabilizer track 206 at a maintained relative distance, the locking assembly 284 prevents the variable encircling turn from being adjusted when the locking assembly 284 is in the first, “VET OFF” mode.

When the locking assembly 284 is in the second mode (FIG. 16B), the stabilizer track lock 286 on the sleeve cart 260 engages stabilizer track 206 and the rack lock 288 on the sleeve cart 260 disengages the teeth 280 of the rack 272. Thus, when the locking assembly 284 is in the second “VET ON” mode, the pusher cart 246 and the rack 272 can move axially relative to the sleeve cart 260, thereby allowing the radius of curvature of the variable encircling turn to be adjusted as shown in FIGS. 7-8. Since the variable encircling turn is adjusted based on relative movement between the independently actuatable shafts, it should be understood that the locking assembly 284 can alternatively be configured such that the sleeve cart 260 can move relative to the pusher cart 246, or both the pusher cart 246 and the sleeve cart 260 can move relative to each other in the second, “VET ON” mode.

In some examples, the locking assembly 284 can be actuated between the first mode and the second mode by rotating the knob 290 between a first rotational position and a second rotational position, respectively. As illustrated in FIG. 15, the first rotational position is a horizontal position (parallel to the axial direction) and the second rotational position is a vertical position (perpendicular to both the axial and lateral directions). Thus, the first rotational position and the second rotational position of the knob 290 are approximately 90 degrees apart. However, in some examples, the first rotational position and the second rotational position can be oriented in any suitable direction relative to the axial direction. The sleeve cart 260 further comprises a marking 291 to indicate the first rotational position and the second rotational position. However, some examples of the locking assembly 284 can comprise a button, a switch, a lever, a slider, a keypad, or any other suitable control interface instead of the knob 290 to actuate the locking assembly 284 between the first mode and the second mode.

The stabilizer track lock 286 (which is also referred to herein as a “rail lock,” a “rail position lock,” or a “track lock”) can, among other things, releasably couple the sleeve cart 260 to the stabilizer track 206 to prevent axial movement of the sleeve cart 260 relative to the stabilizer track 206. The stabilizer track lock 286 comprises a brake pad 292 extending from a bottom portion of the sleeve cart chassis 262 and a cam operably coupling the brake pad 292 to the knob 290. When the knob 290 is rotated from the first rotational position (corresponding to the first or “VET OFF” mode) to the second rotational position (corresponding to the second or “VET ON” mode), the knob 290 rotates the cam, which translates the rotational motion of the knob 290 into linear motion to push the brake pad 292 into frictional engagement with a portion of the stabilizer track 206 (for example, the top portion 208 of the stabilizer track 206).

The rack lock 288 (which is also referred to herein as a “rack gear lock,” a “guide member lock,” or a “guide rail lock”) can, among other things, fixedly couple the sleeve cart 260 to the rack 272 to prevent axial movement of the sleeve cart 260 relative to the rack 272 and the pusher cart 246 fixedly coupled to the rack 272. The rack lock 288 comprises a rotatable disk 293 operably coupled to the knob 290 and a pin 294 extending in the lateral direction from the disk. The pin 294 comprises an off-axis pin extending in the lateral direction from the disk 293 and radially offset from the center of rotation of the disk 293. When the knob 290 is rotated from the second rotational position (corresponding to the second mode or the “VET ON” mode) to the first rotational position (corresponding to the first mode or the “VET OFF” mode), the disk 293 rotates with the knob 90 such that the pin 294 engages at least one of the plurality of teeth 280 of the rack 272. Thus, when the locking assembly 284 is in the first mode, the rack lock 288 prevents the sleeve cart 260 from sliding in the axial direction along the rack 272 relative to the pusher cart 246, thereby preventing the radius of curvature of the variable encircling turn from being adjusted.

In some examples, where the rack 272 is replaced with a rod lacking the plurality of teeth 280, the rack lock 288 can be replaced with a friction brake configured to frictionally engage the rod, thereby locking the sleeve cart 260 to the rod. The friction brake can be operably coupled to the knob 290.

In some examples, the cam of the stabilizer track lock 286 can be coupled to the disk 293 of the rack lock 288 instead of the knob 290.

In some examples, the locking assembly 284 can further comprise a hard stop configured to prevent the knob 290 from being rotated past the first rotational position or the second rotational position.

By coupling both the stabilizer track lock 286 and the rack lock 288 to a single control (such as the knob 290), the user can configure the hub assembly support 244 in one step, action, or motion to adjust the variable encircling turn illustrated in FIGS. 7-8, thereby making the hub assembly support 244 easier and more intuitive to use during the optional variable encircling turn step of the example docking device implantation procedure.

Since the variable encircling turn can be adjusted based on relative movement between the sleeve shaft 182 and the pusher shaft 184, some examples of the hub assembly support 244 feature the locking assembly 284 disposed within the pusher cart 246 rather than the sleeve cart 260, such that the pusher cart 246 is slidingly coupled to the rack 272 and the sleeve cart 260 is fixedly coupled to the rack 272. In some examples, both the pusher cart 246 and the sleeve cart 260 be slidingly coupled to the rack 272, such that both the pusher cart 246 and the sleeve cart 260 can move relative to each other in the second mode (the “VET ON” mode). In such examples, both the pusher cart 246 and the sleeve cart 260 can comprise rack locks 288 and one of (or both) the pusher cart 246 and the sleeve cart 260 can comprise the stabilizer track lock 286.

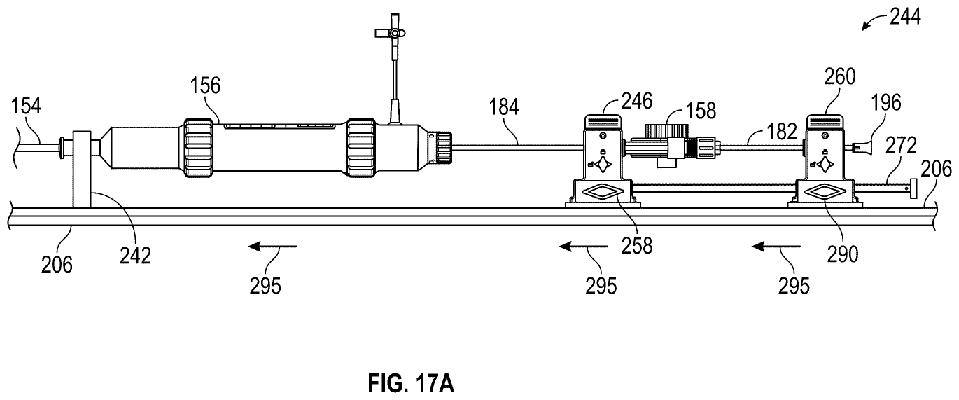

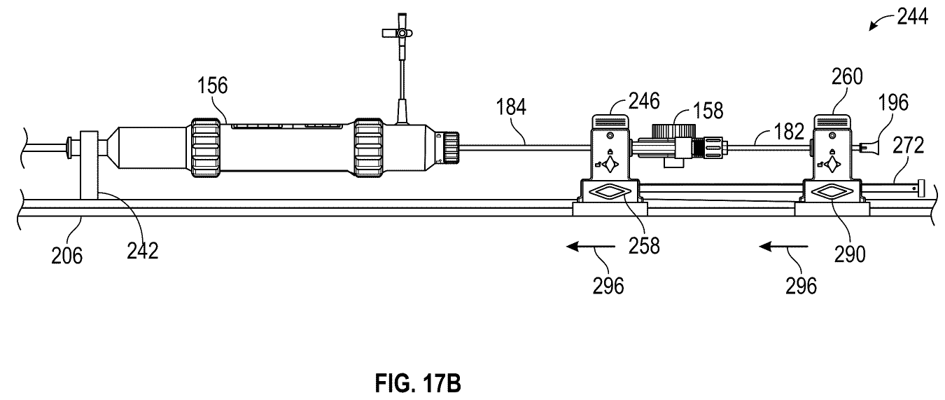

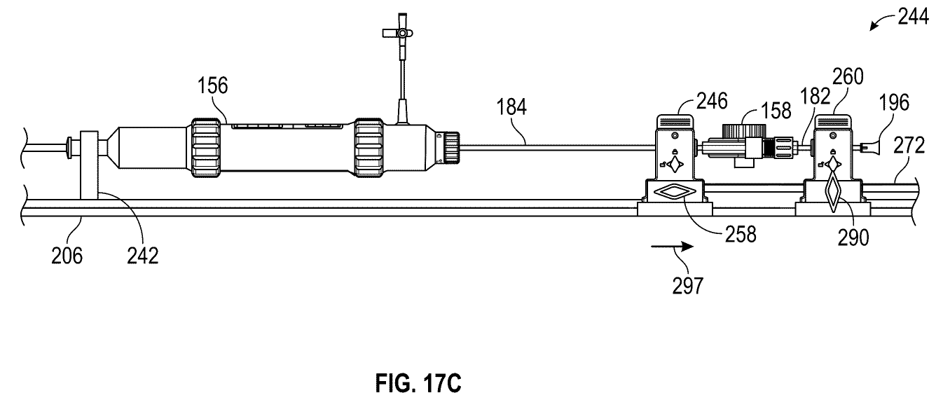

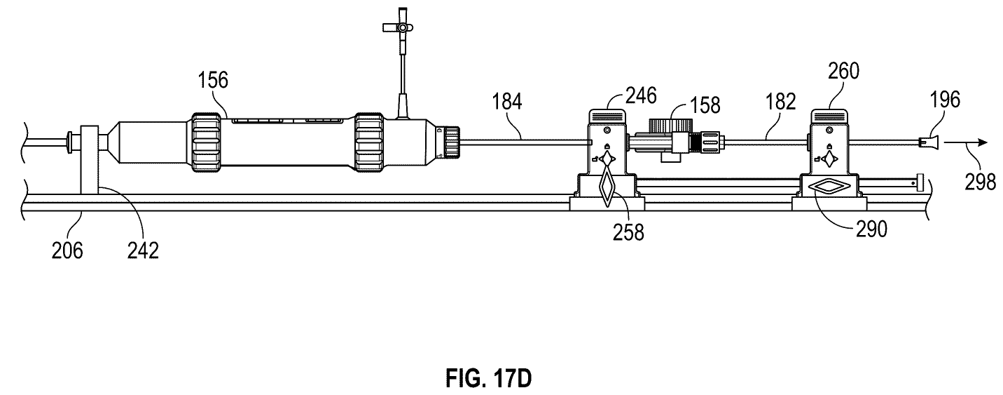

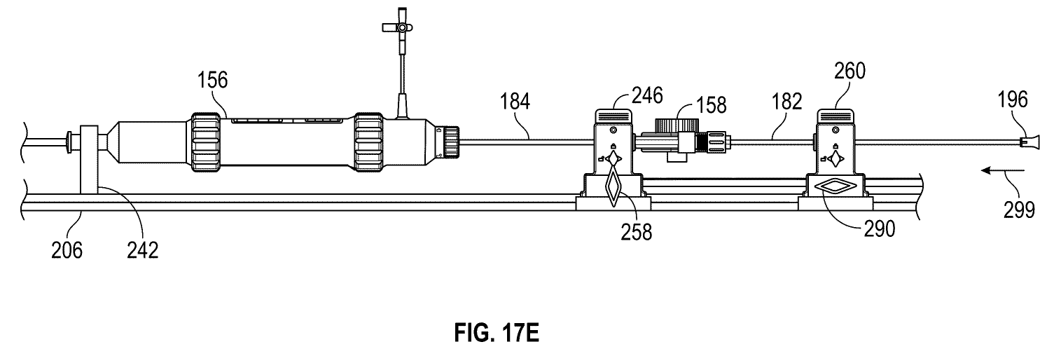

FIGS. 17A-17E illustrate the configuration of the delivery assembly 100 during the example docking device delivery procedure illustrated in FIGS. 5-11. More specifically, FIGS. 17A-17E illustrate the relative axial positions of the handle 156, the hub assembly 158, the hub assembly support 244, and the sleeve handle 196 during various stages of the example procedure. Although the docking device delivery assembly 100 can further comprise other components such as the guide catheter 30, these components are omitted from FIGS. 17A-17E for clarity.

FIG. 17A illustrates the configuration of the docking device delivery assembly 100 during the stage of the example docking device delivery procedure illustrated in FIG. 5. After the guide catheter 30 is deployed, the handle 156, the pusher cart 246, and the sleeve cart 260 are advanced in unison along the stabilizer track 206 in the axial direction (indicated by arrow 295). Since the delivery shaft 154 is coupled to the handle 156, the sleeve shaft 182 is coupled to the sleeve cart 260 (for example, via clamp 264), and the pusher shaft 184 is coupled to the pusher cart 246 (for example, via clamp 250), advancing the handle 156, the pusher cart 246, and the sleeve cart 260 similarly advance the delivery shaft 154, the pusher shaft 184, and the sleeve shaft 182 in unison towards the target implantation site. During this stage, the locking assembly 284 is in the first mode (indicated by the horizontal orientation of knob 290) to lock the sleeve cart 260 to the rack 272 and to unlock the sleeve cart 260 from the stabilizer track 206, thereby maintaining a constant axial separation between the pusher cart 246 and the sleeve cart 260 as the pusher cart 246 and the sleeve cart 260 are slid along the stabilizer track 206. In some examples where the pusher cart 246 further comprises the stabilizer track lock 257, the stabilizer track lock 257 can be unlocked (as indicated by the horizontal orientation of the knob 258) to allow the pusher cart 246 to freely slide along the stabilizer track 206.

FIG. 17B illustrates the configuration of the docking device delivery assembly 100 during the stage of the example docking device delivery procedure illustrated in FIG. 6. After the delivery shaft 154 is advanced to the native mitral valve 16, the sleeve shaft 182 and the pusher shaft 184 are advanced in unison out of the delivery shaft 154 by advancing the pusher cart 246 and the sleeve cart 260 in unison along the stabilizer track 206 in the distal direction (indicated by arrow 296). The handle 156 remains in a fixed axial position relative to the stabilizer track 206. During this stage, the locking assembly 284 is in the first mode (indicated by the horizontal orientation of knob 290) to lock the sleeve cart 260 to the rack 272 and to unlock the sleeve cart 260 from the stabilizer track 206, thereby maintaining constant axial separation between the pusher cart 246 and the sleeve cart 260 as the pusher cart 246 and the sleeve cart 260 are slid along the stabilizer track 206. In some examples where the pusher cart 246 further comprises the stabilizer track lock 257, the stabilizer track lock 257 can be unlocked (as indicated by the horizontal orientation of the knob 258) to allow the pusher cart 246 to freely slide along the stabilizer track 206.

FIG. 17C illustrates the configuration of the delivery assembly 100 during the optional stage of the example docking device delivery procedure illustrated in FIGS. 7-8, when the variable encircling turn is adjusted. During this stage, the locking assembly 284 is actuated from the first mode (the “VET OFF” mode) to the second mode (the “VET ON” mode) to disengage the sleeve cart 260 from the rack 272 and lock and/or fixedly couple the sleeve cart 260 to the stabilizer track 206. Once the sleeve cart 260 and the rack 272 are disengaged, the pusher cart 246 is moved along the stabilizer track 206 in the proximal direction (indicated by arrow 297) relative to the sleeve cart 260 to retract the pusher shaft 184 relative to the sleeve shaft 182. In some examples where the pusher cart 246 further comprises the stabilizer track lock 257, the stabilizer track lock 257 can be unlocked (as indicated by the horizontal orientation of the knob 258) to allow the pusher cart 246 to freely slide along the stabilizer track 206 during the adjustment of the variable encircling turn. In some examples, the pusher cart 246 can be moved along the stabilizer track 206 in the distal direction (opposite the direction of arrow 297) relative to the sleeve cart 260 to advance the pusher shaft 184 relative to the sleeve shaft 182, thereby decreasing the radius of curvature of the variable encircling turn.

It should be understood that the variable encircling turn is adjusted based on relative movement between the sleeve shaft 182 and the pusher shaft 184. Thus, although FIG. 17C illustrates the pusher cart 246 being moved in the axial direction relative to the sleeve cart 260, some examples of this stage can comprise moving the sleeve cart 260 moving in the distal direction (opposite the direction of arrow 297). In such examples, the pusher cart 246 can be locked to the stabilizer track 206. Alternatively, some examples of this stage can comprise moving both the pusher cart 246 and the sleeve cart 260 relative to each other, such that the pusher cart 246 is moved in the proximal direction (in the direction of arrow 297) and the sleeve cart 260 is moved in the distal direction (opposite the direction of arrow 297). In such examples, neither the pusher cart 246 nor the sleeve cart 260 are locked to the stabilizer track 206.

FIG. 17D illustrates the configuration of the docking device delivery assembly 100 during the optional stage of the example docking device delivery procedure illustrated in FIG. 9, when the sleeve shaft 182 is retracted to unsheathe the guard member 180. During this stage, the sleeve handle 196 is independently actuated by removing or decoupling the sleeve handle 196 from the sleeve cart 260 (for example, by removing the sleeve handle from the clamp 264) and moving the sleeve handle 196 in the proximal direction (indicated by arrow 298) relative to the handle 156, the hub assembly 158, and the hub assembly support 244. During this stage, the locking assembly 284 is actuated from the second mode (the “VET ON” mode) to the first mode (the “VET OFF” mode, indicated by the horizontal orientation of the knob 290) to lock the sleeve cart 260 to the rack 272, thereby preventing the pusher cart 246 from moving in the axial direction relative to the sleeve cart 260. In some examples where the pusher cart 246 further comprises the stabilizer track lock 257, the stabilizer track lock 257 can be locked (as indicated by the vertical orientation of the knob 258) to lock the pusher cart 246 to the stabilizer track 206, thereby preventing the pusher shaft 184 from moving during this stage.

Since the guard member 180 is unsheathed by relative movement between the sleeve shaft 182 and the pusher shaft 184, some examples of the stage illustrated in FIG. 17D can comprise moving the hub assembly 158 in the distal direction (opposite the direction of arrow 298) relative to the sleeve handle 196. For example, the locking assembly 284 can be set to the second mode, such that the pusher cart 246 and the hub assembly 158 can be moved in the distal direction relative to the sleeve cart 260 and the sleeve handle 196. In some examples, the pusher shaft 184 can be decoupled from the pusher cart 246 such that the hub assembly 158 can be freely moved in the axial direction relative to the sleeve shaft 182 and sleeve handle 196. In some examples, the hub assembly support 244 can be configured such that both the pusher cart 246 and the sleeve cart 260 move relative to each other during this stage to unsheathe the guard member 180. In some examples where the docking device delivery procedure includes the optional atrial reveal step, the handle 156 can be retracted in the proximal direction while the pusher cart 246 is locked to the stabilizer track 206.

FIG. 17E illustrates the configuration of the docking device delivery assembly 100 during the optional stage of the example docking device delivery procedure illustrated in FIG. 10, when the guard member 180 is axially foreshortened by advancing the distal end portion 186 of the sleeve shaft 182 in the distal direction to exert a force against the proximal end portion 193 of the guard member 180. The distal end portion 186 of the sleeve shaft 182 is advanced by advancing the sleeve handle 196 in the distal direction (indicated by arrow 299). In some examples where the pusher cart 246 further comprises the stabilizer track lock 257, the stabilizer track lock 257 can be locked (as indicated by the vertical orientation of the knob 258) to lock the pusher cart 246 to the stabilizer track 206, thereby preventing the pusher shaft 184 from moving during this stage.

Since the guard member 180 is axially foreshortened by relative movement between the sleeve shaft 182 and the guard member 180 coupled to the pusher shaft 184, some examples of the stage illustrated in FIG. 17E can comprise moving the hub assembly 158 in the proximal direction (opposite the direction of arrow 299) relative to the sleeve handle 196. For example, the locking assembly 284 can be set to the second mode, such that the pusher cart 246 and the hub assembly 158 can be moved in the proximal direction relative to the sleeve cart 260 and the sleeve handle 196. In some examples, the pusher shaft 184 can be decoupled from the pusher cart 246 such that the hub assembly 158 can be freely moved in the axial direction relative to the sleeve shaft 182 and sleeve handle 196. In some examples, the hub assembly support 244 can be configured such that both the pusher cart 246 and the sleeve cart 260 move relative to each other during this stage to axially foreshorten the guard member 180.

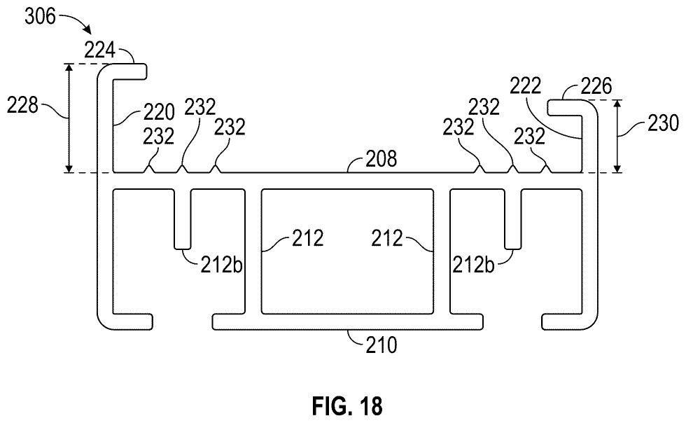

FIG. 18 shows a stabilizer track 306, according to a second example.

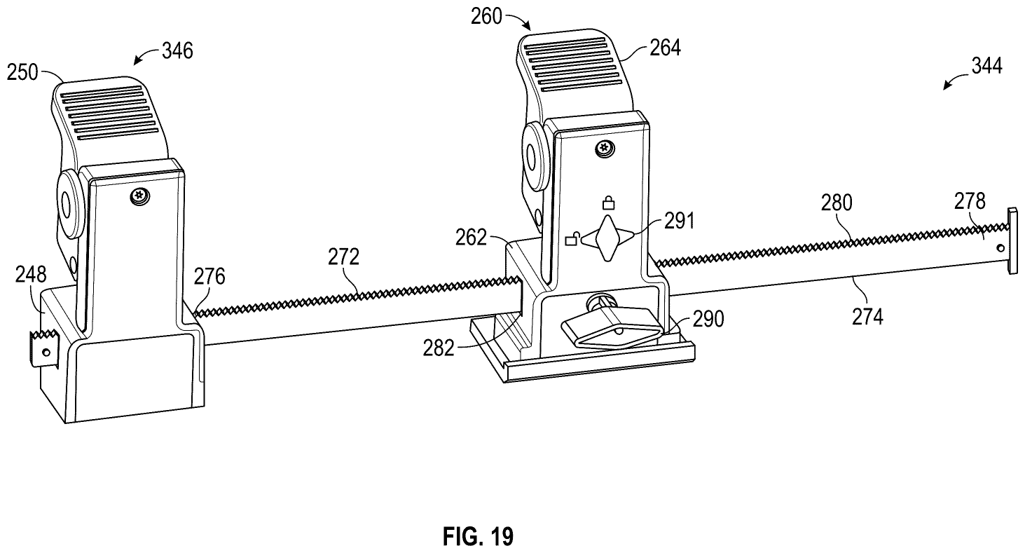

FIG. 19 shows a hub assembly support 344, according to a second example. One exemplary difference between the hub assembly support 344 and the hub assembly support 244 of FIG. 15 is that a pusher cart 346 is cantilevered from the sleeve cart 260 and is not directly coupled to the stabilizer track 206. Thus, the pusher cart 346 lacks the first and second rail engagement flanges 254, 256 because the pusher cart 346 is not configured to directly couple to the stabilizer track 206. In some examples, the design of the cantilevered hub assembly support 344 can be inverted such that the sleeve cart 260 can be cantilevered from the pusher cart 346. In some examples, cantilevering one of the pusher cart 346 and the sleeve cart 260 can beneficially reduce the length of the stabilizer track 206 because the hub assembly support 344 is coupled to the stabilizer track 206 at only one point.

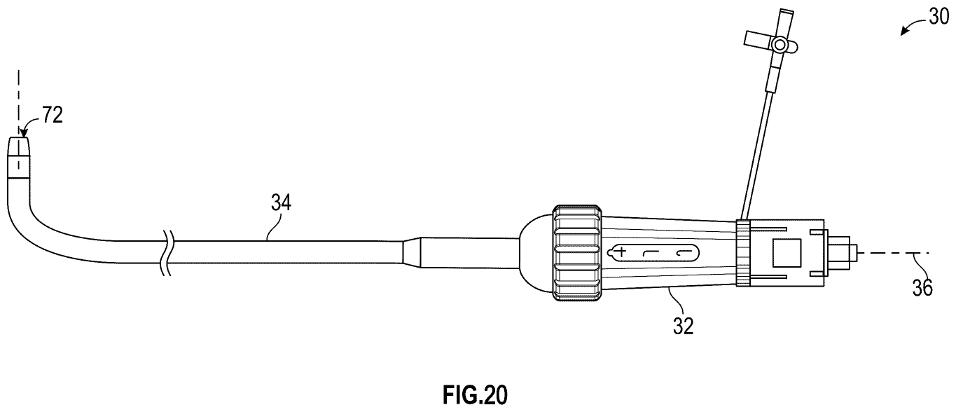

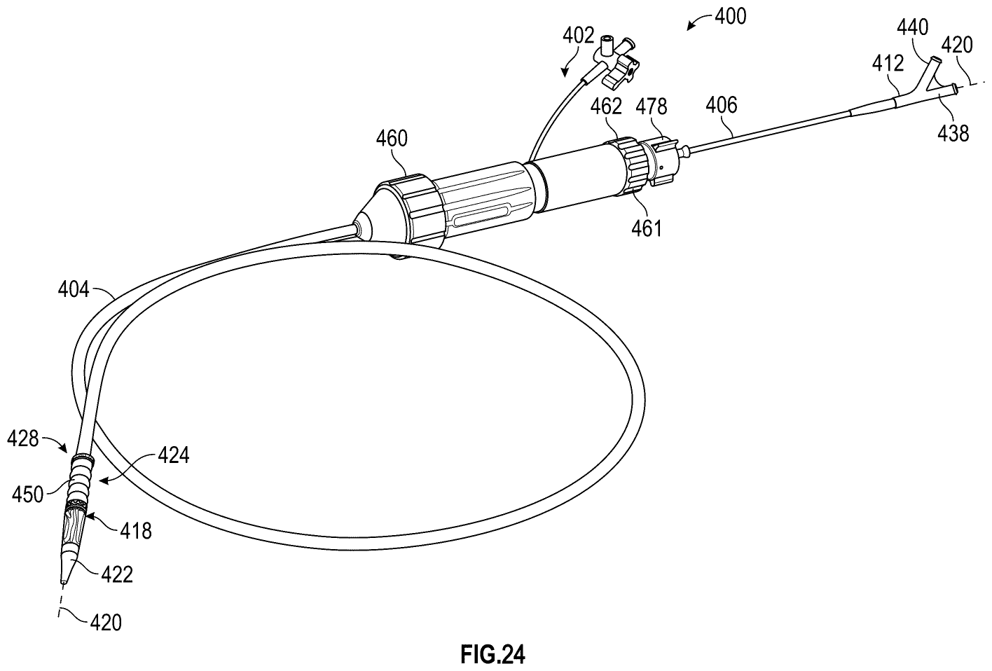

FIG. 20 illustrates the guide catheter 30 (which may be referred to herein as an “introducer device”), according to one example. In some examples, the guide catheter 30 can be used in a prosthetic valve implantation procedure, as described above with reference to FIGS. 1-4. In some examples, the guide catheter 30 can be used in a docking device implantation procedure, as described with reference to FIGS. 5-11. The guide catheter 30 is be configured to be inserted into a patient's vasculature and receive an implant catheter (and/or other delivery apparatus) therein to introduce the implant catheter into the patient's vasculature and at least partially guide the implant catheter to a target implantation site. Examples of implant catheters for prosthetic medical devices (referred to below as “delivery apparatus 150” and “delivery apparatus 400”) that can be received within the guide catheter 30 are shown in FIGS. 13 and 24 respectively.

The guide catheter 30 in the illustrated example comprises the handle 32, the catheter shaft 34 extending distally from the handle 32, and a longitudinal axis 36. In some examples, the catheter shaft 34 can extend proximally into the handle 32. In some examples, the catheter shaft 34 can be coupled to a distal end portion of the handle 32. The handle 32 comprises a catheter handle lumen (not pictured) extending through the length of the handle 32. The catheter handle lumen is axially aligned with the catheter shaft lumen and coupled to a distal end portion of the catheter shaft 34, such that the delivery shaft 154, the sleeve shaft 182, and the pusher shaft 184 can extend through the catheter handle lumen and the catheter shaft lumen. In some examples, the catheter shaft lumen and the delivery shaft 154 can be aligned with the longitudinal axis 36.

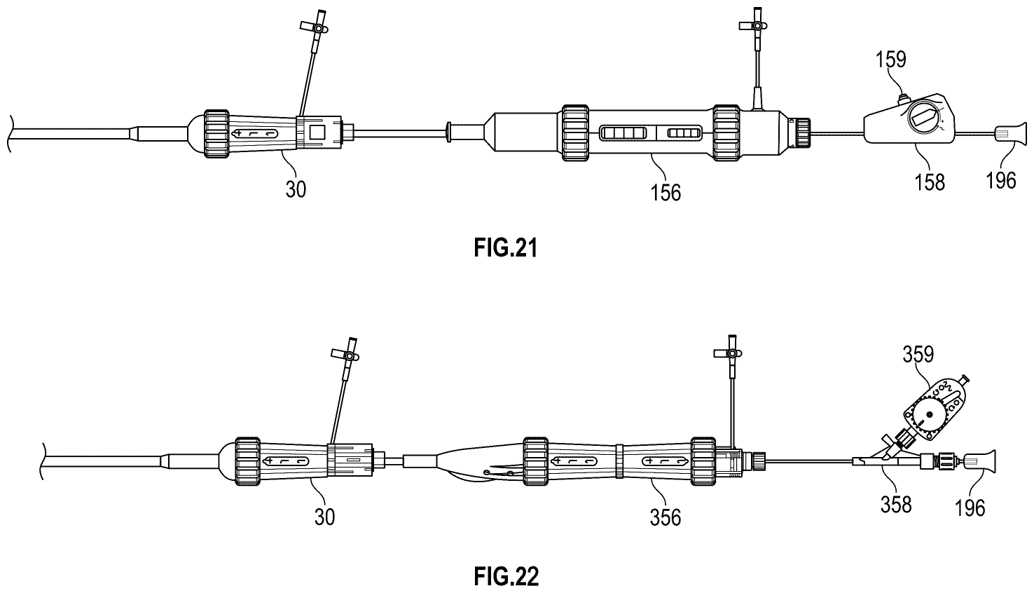

FIG. 21 illustrates the guide catheter 30 coupled to the delivery apparatus 150, according to one example.

FIG. 22 illustrates the guide catheter 30 coupled to a docking device delivery apparatus 350, according to a second example. The docking device delivery apparatus 350 comprises a handle 356 (which can be similar to the handle 156), a hub assembly 358 (which can be similar to the hub assembly 158) comprising a suture lock assembly 359, and the sleeve handle 196.

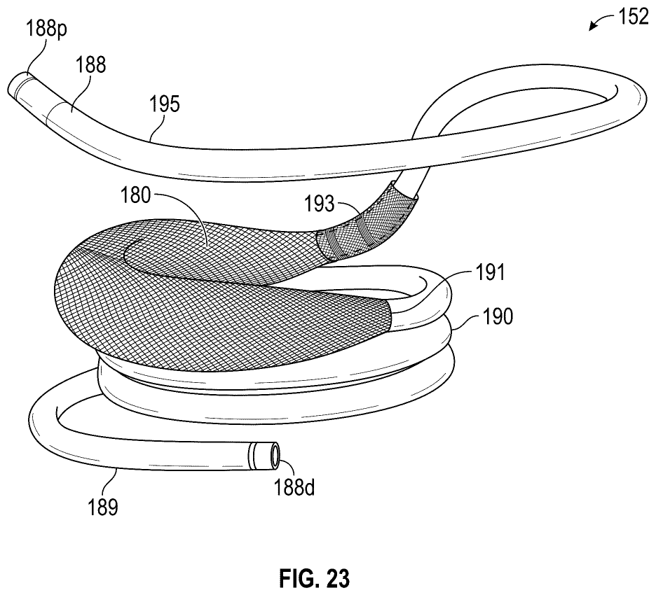

FIG. 23 illustrates the docking device 152, according to one example. As depicted in FIG. 23, the docking device 152 in its deployed coiled configuration is configured to receive and secure a prosthetic valve (such as the prosthetic heart valve 62) within the docking device 152, thereby securing the prosthetic valve at the annulus of the native mitral valve 16.

The docking device 152 comprises a coil 188. In some examples, the coil 188 can include a shape memory material (for example, nickel titanium alloy or “Nitinol”) such that the docking device 152 (and the coil 188) can move from a substantially straight configuration (or delivery configuration) when disposed within the delivery shaft 154 to a helical, deployed configuration after being removed from the delivery shaft 154.

The coil 188 has a proximal end 188p and a distal end 188d (which also respectively define the proximal and distal ends of the docking device 152). When being disposed within the delivery shaft 154 (for example, during delivery of the docking device 152 into the vasculature of a patient), a body of the coil 188 between the proximal end 188p and distal end 188d forms a generally straight delivery configuration (without any coiled or looped portions, but can be flexed or bent) so as to maintain a small radial profile when moving through a patient's vasculature. After being removed from the delivery shaft 154 and deployed at an implant position, the coil 188 moves from the delivery configuration to the helical deployed configuration and wraps around native tissue adjacent the implant position. For example, when implanting the docking device at the location of a native valve, the coil 188 can be configured to surround native leaflets of the native valve (and the chordae tendineae that connects native leaflets to adjacent papillary muscles).

The coil 188 in the deployed coiled configuration includes the docking device leading turn 189, the central region 190, and a stabilization turn 195 (or “stabilization coil”) around a central longitudinal axis.

In the deployed coiled configuration, the central region 190 comprises one or more helical turns formed around a central longitudinal axis of the docking device 152, wherein the helical turns have substantially equal radii of curvature configured to encircle the leaflets 24 of the native mitral valve 16. The docking device leading turn 189 extends from a distal end of the central region 190 and has a radius of curvature greater than the radius of curvature of the helical turns of the central region 190. In some examples, the radius of curvature of the docking device leading turn 189 of the docking device 152 is equal to a second inner radius of curvature, wherein the second inner radius of curvature is less than the first inner radius of curvature of the sleeve shaft leading turn 187.

The stabilization turn 195 extends from a proximal end of the central region 190 and has a radius of curvature greater than the radius of curvature of the central region 190, in the illustrated example. Alternatively, the stabilization turn 195 can have a radius of curvature that is equal, approximately equal, or less than the radius of curvature of the central region 190 (as opposed to larger), and/or the stabilization turn can comprise less of a full turn than depicted in FIG. 23.

In some examples, the docking device 152 can further comprise the guard member 180 disposed on the coil 188. The guard member is configured to reduce the possibility of paravalvular leakage between the native mitral valve 16 and the prosthetic heart valve. In some examples, the guard member 180 can comprise a braided portion disposed between the distal end portion 191 and the proximal end portion 193 of the guard member 180. The braided portion is configured to foreshorten into a deployed configuration when the proximal end portion 193 is forced in a distal direction, wherein the braided portion has an increased radial thickness in the foreshortened, deployed configuration.

Further details of the docking device and its variants are described in PCT Publication No. WO2022/087336, which is incorporated by reference herein in its entirety.

FIG. 24 illustrates a delivery apparatus 400 (which can also be referred to here as an “implant catheter” and/or a “prosthetic heart valve delivery apparatus”) that can be used to implant an expandable prosthetic heart valve, according to one example. In some examples, the delivery apparatus 400 is specifically adapted for use in introducing a prosthetic heart valve into a heart. For example, the delivery apparatus 400 can be used as the prosthetic valve delivery apparatus 60 in a prosthetic valve implantation procedure, as described above with reference to FIG. 3A.

The delivery apparatus 400 in the illustrated example of FIG. 24 is a balloon catheter comprising a handle 402 and a steerable, outer shaft 404 extending distally from the handle 402. The delivery apparatus 400 can further comprise an intermediate shaft 406 (which also may be referred to as a balloon shaft) that extends proximally from the handle 402 and distally from the handle 402, the portion extending distally from the handle 402 also extending coaxially through the outer shaft 404. In some examples, the delivery apparatus 400 can further comprise an inner shaft extending distally from the handle 402 coaxially through the intermediate shaft 406 and the outer shaft 404 and proximally from the handle 402 coaxially through the intermediate shaft.

The outer shaft 404 and the intermediate shaft 406 can be configured to translate (for example, move) longitudinally, along a central longitudinal axis 420 of the delivery apparatus 400, relative to one another to facilitate delivery and positioning of a prosthetic valve at an implantation site in a patient's body.

The intermediate shaft 406 can include a proximal end portion that extends proximally from a proximal end of the handle 402, to an adaptor 412. The adaptor 412 can include a first port 438 configured to receive a guidewire therethrough and a second port 440 configured to receive fluid (for example, inflation fluid) from a fluid source. The second port 440 can be fluidly coupled to an inner lumen of the intermediate shaft 406.

In some examples, the intermediate shaft 406 can further include a distal end portion that extends distally beyond a distal end of the outer shaft 404 when a distal end of the outer shaft 404 is positioned away from an inflatable balloon 418 of the delivery apparatus 400. A distal end portion of the inner shaft can extend distally beyond the distal end portion of the intermediate shaft 406 toward or to a nose cone 422 at a distal end of the delivery apparatus 400.

In some examples, a distal end of the balloon 418 can be coupled to a distal end of the delivery apparatus 400, such as to the nose cone 422 (as shown in FIG. 24), or to an alternate component at the distal end of the delivery apparatus 400 (for example, a distal shoulder). An intermediate portion of the balloon 418 can overlay a valve mounting portion 424 of a distal end portion of the delivery apparatus 400 and a distal end portion of the balloon 418 can overly a distal shoulder of the delivery apparatus 400. As shown in FIG. 24, a prosthetic heart valve 450 can be mounted around the balloon 418, at the valve mounting portion 424 of the delivery apparatus 400, in a radially compressed state. The prosthetic heart valve 450 can be configured to be radially expanded by inflation of the balloon 418 at a native valve annulus, as described above with reference to FIG. 3A.

A balloon shoulder assembly of the delivery apparatus 400, which includes the distal shoulder, is configured to maintain the prosthetic heart valve 450 (or other medical device) at a fixed position on the balloon 418 during delivery through the patient's vasculature.

The outer shaft 404 can include a distal tip portion 428 mounted on its distal end. In some examples, the outer shaft 404 and the intermediate shaft 406 can be translated axially relative to one another to position the distal tip portion 428 adjacent to a proximal end of the valve mounting portion 424, when the prosthetic valve 450 is mounted in the radially compressed state on the valve mounting portion 424 (as shown in FIG. 24) and during delivery of the prosthetic valve to the target implantation site. As such, the distal tip portion 428 can be configured to resist movement of the prosthetic valve 450 relative to the balloon 418 proximally, in the axial direction, relative to the balloon 418, when the distal tip portion 428 is arranged adjacent to a proximal side of the valve mounting portion 424.

An annular space can be defined between an outer surface of the inner shaft and an inner surface of the intermediate shaft 406 and can be configured to receive fluid from a fluid source via the second port 440 of the adaptor 412. The annular space can be fluidly coupled to a fluid passageway formed between the outer surface of the distal end portion of the inner shaft and an inner surface of the balloon 418. As such, fluid from the fluid source can flow to the fluid passageway from the annular space to inflate the balloon 418 and radially expand and deploy the prosthetic valve 450.

An inner lumen of the inner shaft can be configured to receive a guidewire therethrough, for navigating the distal end portion of the delivery apparatus 400 to the target implantation site.

The handle 402 can include a steering mechanism configured to adjust the curvature of the distal end portion of the delivery apparatus 400. In the illustrated example, for example, the handle 402 includes an adjustment member, such as the illustrated rotatable knob 460, which in turn is operatively coupled to the proximal end portion of a pull wire. The pull wire can extend distally from the handle 402 through the outer shaft 404 and has a distal end portion affixed to the outer shaft 304 at or near the distal end of the outer shaft 404. Rotating the knob 460 can increase or decrease the tension in the pull wire, thereby adjusting the curvature of the distal end portion of the delivery apparatus 400. Further details on steering or flex mechanisms for the delivery apparatus can be found in U.S. Pat. No. 9,339,384, as previously incorporated by reference above.

The handle 402 can further include an adjustment mechanism 461 including an adjustment member, such as the illustrated rotatable knob 462, and an associated locking mechanism including another adjustment member, configured as a rotatable knob 478. The adjustment mechanism 361 is configured to adjust the axial position of the intermediate shaft 406 relative to the outer shaft 404 (for example, for fine positioning at the implantation site).

Prosthetic valves disclosed herein (for example, prosthetic heart valve 450, prosthetic heart valve 62, etc.) can be radially compressible and expandable between a radially compressed state and a radially expanded state. Thus, the prosthetic valves can be crimped on or retained by an implant delivery apparatus (for example, delivery apparatus 400, prosthetic delivery apparatus 60, etc.) in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

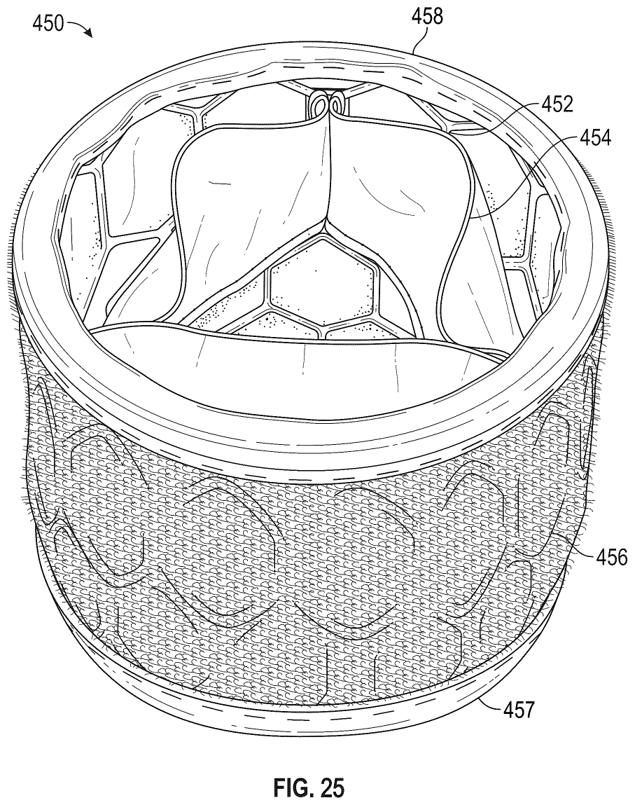

FIG. 25 illustrates the prosthetic valve 450 in a radially expanded position. The prosthetic valve 450 can be used as the prosthetic heart valve 62 in a prosthetic valve implantation procedure, as described above with reference to FIGS. 1-4. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (for example, docking device 152, etc.) that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic valve 450 can be used as the prosthetic heart valve 62 in a prosthetic valve implantation procedure, as described above with reference to FIGS. 1-4. As shown in FIG. 25, the prosthetic valve 450 can include a frame 452 and a plurality of leaflets 454 can be situated at least partially within the frame 452. The prosthetic valve 450 can also include an outer covering 456 situated about the frame 452. As shown in FIG. 25, the prosthetic valve 450 includes an inflow end 457 and an outflow end 458. The terms “inflow” and “outflow” are related to the normal direction of blood flow (for example, antegrade blood flow) through the prosthetic valve 450. For example, the leaflets 454 can allow blood flow through the valve 450 in a direction from the inflow end 457 to the outflow end 458 and prevent the reverse flow (for example, prevent flow in a direction from the outflow end 458 to the inflow end 457).

The frame 452 can be made of any of various suitable plastically-expandable materials (for example, stainless steel, etc.) or self-expanding materials (for example, Nitinol) as known in the art. When constructed of a plastically-expandable material, the frame 452 (and thus the valve 450) can be crimped to a radially compressed state on a delivery catheter and then expanded inside a patient by an inflatable balloon or equivalent expansion mechanism. When constructed of a self-expandable material, the frame 452 (and thus the valve 450) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 452) include, metal alloys, polymers, or combinations thereof. Example metal alloys can comprise one or more of the following: nickel, cobalt, chromium, molybdenum, titanium, or other biocompatible metal. In some examples, the frame 452 can comprise stainless steel. In some examples, the frame 452 can comprise cobalt-chromium. In some examples, the frame 452 can comprise nickel-cobalt-chromium. In some examples, the frame 452 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The outer covering 456 can be wholly or partly formed of any suitable biological material, synthetic material (for example, any of various polymers), or combinations thereof. In some examples, the outer covering 456 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer covering 456 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the outer covering 456 can comprise a non-textile or non-fabric material, such as a film including any of a variety of crystalline or semi-crystalline polymeric materials, such as PTFE, PET, polypropylene, polyamide, polyetheretherketone (PEEK), polyurethane (such as thermoplastic polyurethane (TPU)), etc. In some examples, the outer covering 456 can comprise a sponge material or foam, such as polyurethane foam. In some examples, the outer covering 456 can comprise natural tissue, such as pericardium (for example, bovine pericardium, porcine pericardium, equine pericardium, or pericardium from other sources).

Further details of the prosthetic heart valve and its variants are described in U.S. Pat. No. 11,185,406, which is incorporated by reference herein in its entirety.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A docking device delivery assembly comprising:
a delivery apparatus comprising:
   a first shaft extending in a distal direction; and
   a second shaft coaxial with the first shaft; and
a support for the delivery apparatus, the support comprising:
   a track extending in an axial direction;
   a first cart slidingly coupled to the track, wherein the first cart is configured to couple to the first shaft of the delivery apparatus;
   a guide member fixedly coupled to the first cart; and
   a second cart actuatable between a first mode and a second mode, wherein:
      the second cart is configured to couple to the second shaft,
      in the first mode, the second cart is slidingly coupled to the track and is fixedly coupled to the guide member, and
      in the second mode, the second cart is fixedly coupled to the track and is slidingly coupled to the guide member.
Example 2. The docking device delivery assembly of any example herein, particularly Example 1, wherein the second cart further comprises a locking assembly configured to selectively engage the track and the guide member.
Example 3. The docking device delivery assembly of any example herein, particularly Example 2, wherein the locking assembly comprises a track lock.
Example 4. The docking device delivery assembly of any example herein, particularly Example 3, wherein the track lock is locked to the track in the second mode and unlocked from the track in the first mode.
Example 5. The docking device delivery assembly of any example herein, particularly Example 3, wherein the track lock comprises a brake pad configured to frictionally engage the track.
Example 6. The docking device delivery assembly of any example herein, particularly Example 2, wherein the locking assembly comprises a guide member lock.
Example 7. The docking device delivery assembly of any example herein, particularly Example 6, wherein the guide member lock is locked to the guide member in the first mode and unlocked from the guide member in the second mode.
Example 8. The docking device delivery assembly of any example herein, particularly Example 6, wherein the guide member comprises a plurality of teeth extending along the guide member in the axial direction, and wherein the guide member lock comprises a pin configured to engage at least one of the plurality of teeth.
Example 9. The docking device delivery assembly of any example herein, particularly Example 6, wherein the guide member lock comprises a friction brake configured to frictionally engage the guide member.
Example 10. A support for use with a delivery apparatus, the support comprising:
a track extending in an axial direction;
a first cart configured to slide along the track and couple to a first portion of the delivery apparatus;
a guide rail fixedly coupled to an axial end portion of the first cart and extending in the axial direction; and
a second cart configured to slide along the track and couple to a second portion of the delivery apparatus, the second cart comprising:
   a locking assembly actuatable between a first mode and a second mode, wherein:
      in the first mode, the locking assembly engages the guide rail and disengages the track, and
      in the second mode, the locking assembly disengages the guide rail and engages the track.
Example 11. The support of any example herein, particularly Example 10, wherein the locking assembly comprises a track lock and a guide rail lock.
Example 12. The support of any example herein, particularly Example 11, wherein the track lock comprises a brake pad configured to frictionally engage the track.
Example 13. The support of any example herein, particularly Example 11, wherein the guide rail comprises a plurality of teeth extending along the guide rail in the axial direction, and wherein the guide rail lock comprises a pin configured to engage at least one of the plurality of teeth.
Example 14. The support of any example herein, particularly Example 11, wherein the guide rail lock comprises a friction brake configured to frictionally engage the guide rail.
Example 15. The support of any example herein, particularly Example 11, wherein the locking assembly further comprises a rotatable knob to actuate the locking assembly between the first mode and the second mode.
Example 16. The support of any example herein, particularly Example 15, wherein the rotatable knob is configured to actuate both the track lock and the guide rail lock.
Example 17. The support of any example herein, particularly any one of Examples 10-16, wherein the first cart further comprises a clamp configured to receive the first portion of the delivery apparatus.
Example 18. The support of any example herein, particularly any one of Examples 10-17, wherein the first portion of the delivery apparatus is a pusher shaft.
Example 19. The support of any example herein, particularly any one of Examples 10-18, wherein the second cart further comprises a clamp configured to receive the second portion of the delivery apparatus.
Example 20. The support of any example herein, particularly any one of Examples 10-19, wherein the second portion of the delivery apparatus is a sleeve shaft.
Example 21. A support assembly comprising:
a rack gear comprising a first axial end portion and a second axial end portion;
a first cart fixedly coupled to the first axial end portion;
a second cart is configured to slide along the rack gear between the first axial end portion and the second axial end portion; and
a locking assembly comprising:
   a lock configured to lock the second cart to the rack gear; and
   a rotatable knob configured to actuate the locking assembly.
Example 22. The support assembly of any example herein, particularly Example 21, wherein the rack gear comprises a plurality of teeth extending in an axial direction.
Example 23. The support assembly of any example herein, particularly Example 22, wherein the locking assembly comprises a disk rotatable between a locked configuration and an unlocked configuration and a pin extending from the disk, wherein the pin is configured to engage at least one of the plurality of teeth.
Example 24. The support assembly of any example herein, particularly Example 23, wherein the pin is an off-axis pin extending from a lateral side of the disk.
Example 25. The support assembly of any example herein, particularly any one of Examples 21-24, wherein the rotatable knob is rotatable between a first rotational position and a second rotational position.
Example 26. The support assembly of any example herein, particularly Example 25, wherein the support assembly further comprises a hard stop to prevent the rotatable knob from rotating past at least one of the first rotational position and the second rotational position.
Example 27. The support assembly of any example herein, particularly any one of Examples 21-26, wherein the support assembly further comprises a hard stop on the second axial end portion of the rack gear.
Example 28. The support assembly of any example herein, particularly any one of Examples 21-27, wherein the first cart is cantilevered from the second cart.
Example 29. A stabilizer assembly for a delivery apparatus, the stabilizer assembly comprising:
a track extending in an axial direction;
a first post coupled to the track;
a rack gear comprising a proximal end portion and a distal end portion, wherein the distal end portion is fixedly coupled to a proximal end portion of the first post;
a second post coupled to the track, wherein the second post comprises:
   a slot extending in the axial direction through the second post, wherein the slot is configured to receive at least a portion of the rack gear; and
   a locking assembly actuatable between a first position and a second position, wherein:
      in the first position, the locking assembly disengages the rack gear and engages the track, and
      in the second position, the locking assembly engages the rack gear and disengages the track.
Example 30. The stabilizer assembly of any example herein, particularly Example 29, wherein the track comprises a first web having a first height and a second web having a second height.
Example 31. The stabilizer assembly of any example herein, particularly Example 30, wherein the first height is greater than the second height.
Example 32. The stabilizer assembly of any example herein, particularly any one of Examples 29-31, wherein the track comprises a unitary structure.
Example 33. The stabilizer assembly of any example herein, particularly any ne of Examples 29-32, wherein rail is an extruded structure.
Example 34. The stabilizer assembly of any example herein, particularly any one of Examples 29-33, wherein the rail is formed of ABS.
Example 35. The stabilizer assembly of any example herein, particularly any one of Examples 29-34, wherein the rail is formed of aluminum.
Example 36. The stabilizer assembly of any example herein, particularly any one of Examples 29-35, wherein the rail comprises a top surface, a bottom surface, and at least one stiffening web extending therebetween.
Example 37. The stabilizer assembly of any example herein, particularly Example 36, wherein the at least one stiffening web comprises a top end portion coupled to the top surface, a bottom end portion coupled to the bottom surface, and an intermediate portion.
Example 38. The stabilizer assembly of any example herein, particularly Example 37, wherein the intermediate portion has a C-shaped axial cross section.
Example 39. The stabilizer assembly of any example herein, particularly Example 37, wherein the intermediate portion has an O-shaped axial cross section.
Example 40. A hub assembly support for a delivery apparatus, comprising:
a first post configured to slidingly couple to a track extending in an axial direction;
a guide rail fixedly coupled to an axial end portion of the first post, wherein the guide rail extends from the axial end portion of the first post in the axial direction; and
a second post slidingly coupled to both the track and the guide rail.
Example 41. The hub assembly support of any example herein, particularly Example 40, wherein the second post further comprises a locking assembly, wherein the locking assembly is configured to selectively engage the track and the guide rail and to restrict relative movement of the first post along the track relative to the second post.
Example 42. A track for a delivery apparatus stabilizer assembly, the track comprising:
a top surface extending along a length of the track;
a bottom surface extending along the length of the track;
a first web extending from the top surface and along the length of the track, wherein the first web has a first height; and
a second web extending from the top surface and along the length of the track, wherein:
   the first web has a second height,
   the first height is different than the second height, and
   the track is formed as a unitary structure.
Example 43. A method of implanting a prosthetic medical device, comprising:
coupling a delivery apparatus to a stabilizer assembly, wherein:
   the delivery apparatus comprises first and second coaxial shafts, wherein the first and second coaxial shafts are independently actuatable, and
   the stabilizer assembly comprises:
      a rail oriented in an axial direction;
      a first post slidably couple to the rail, wherein the first shaft is configured to couple to the first post;
      a second post slidably coupled to the rail, wherein the second shaft is configured to couple to the second post;
      a guide member fixedly coupled to the first post and slidably coupled to the second post; and
      a locking assembly disposed on the second post and configured to selectively engage the rail and the guide member;
sliding the first post and the second post in unison in a distal direction along the rail;
actuating the locking assembly to engage the rail and disengage the guide member; and
sliding the first post in the axial direction relative to the second post.
Example 44. The method of any example herein, particularly Example 43, wherein the method further comprises a step, after sliding the first post in the proximal direction relative to the second post, of actuating the locking assembly to disengage the rail and engage the guide member.
Example 45. The method of any example herein, particularly Example 44, wherein the method further comprises a step, after actuating the locking assembly to disengage the rail and engage the guide member, of disengaging the second shaft from the second post and actuating the second shaft in the proximal direction.
Example 46. The method of any example herein, particularly Example 45, wherein the method further comprises a step, after disengaging actuating the second shaft in the proximal direction, of actuating the second shaft in the distal direction.
Example 47. The method of any example herein, particularly Example 46, wherein the method further comprises a step, after disengaging the second shaft from the second post and actuating the second shaft in the proximal direction and before actuating the second shaft in the distal direction, of actuating a handle of the delivery apparatus in a proximal direction.

The features described herein with regard to any example can be combined with other features described in any one or more of the other examples, unless otherwise stated. For example, any one or more of the features of a hub assembly support can be combined with any one or more features of another hub assembly support. As another example, any one or more features of one docking device delivery apparatus can be combined with any one or more features of another docking device delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A docking device delivery assembly comprising:
a delivery apparatus comprising:
a first shaft extending in a distal direction; and
a second shaft coaxial with the first shaft; and
a support for the delivery apparatus, the support comprising:
a track extending in an axial direction;
a first cart slidingly coupled to the track, wherein the first cart is configured to couple to the first shaft of the delivery apparatus;
a guide member fixedly coupled to the first cart; and
a second cart actuatable between a first mode and a second mode, wherein:
the second cart is configured to couple to the second shaft,
in the first mode, the second cart is slidingly coupled to the track and is fixedly coupled to the guide member, and
in the second mode, the second cart is fixedly coupled to the track and is slidingly coupled to the guide member.

2. The docking device delivery assembly of claim 1, wherein the second cart further comprises a locking assembly configured to selectively engage the track and the guide member.

3. The docking device delivery assembly of claim 2, wherein the locking assembly comprises a track lock.

4. The docking device delivery assembly of claim 3, wherein the track lock is locked to the track in the second mode and unlocked from the track in the first mode.

5. The docking device delivery assembly of claim 3, wherein the track lock comprises a brake pad configured to frictionally engage the track.

6. The docking device delivery assembly of claim 2, wherein the locking assembly comprises a guide member lock.

7. The docking device delivery assembly of claim 6, wherein the guide member lock is locked to the guide member in the first mode and unlocked from the guide member in the second mode.

8. The docking device delivery assembly of claim 6, wherein the guide member comprises a plurality of teeth extending along the guide member in the axial direction, and wherein the guide member lock comprises a pin configured to engage at least one of the plurality of teeth.

9. The docking device delivery assembly of claim 6, wherein the guide member lock comprises a friction brake configured to frictionally engage the guide member.

10. A stabilizer assembly for a delivery apparatus, the stabilizer assembly comprising:
a track extending in an axial direction;
a first post coupled to the track;
a rack gear comprising a proximal end portion and a distal end portion, wherein the distal end portion is fixedly coupled to a proximal end portion of the first post;
a second post coupled to the track, wherein the second post comprises:
a slot extending in the axial direction through the second post, wherein the slot is configured to receive at least a portion of the rack gear; and
a locking assembly actuatable between a first position and a second position, wherein:
in the first position, the locking assembly disengages the rack gear and engages the track, and
in the second position, the locking assembly engages the rack gear and disengages the track.

11. The stabilizer assembly of claim 10, wherein the track comprises a first web having a first height and a second web having a second height.

12. The stabilizer assembly of claim 11, wherein the first height is greater than the second height.

13. The stabilizer assembly of claim 10, wherein rail is an extruded structure and comprises a top surface, a bottom surface, and at least one stiffening web extending therebetween.

14. The stabilizer assembly of claim 13, wherein the at least one stiffening web comprises a top end portion coupled to the top surface, a bottom end portion coupled to the bottom surface, and an intermediate portion.

15. The stabilizer assembly of claim 14, wherein the intermediate portion has a C-shaped axial cross section.

16. A method of implanting a prosthetic medical device, comprising:
coupling a delivery apparatus to a stabilizer assembly, wherein:
the delivery apparatus comprises first and second coaxial shafts, wherein the first and second coaxial shafts are independently actuatable, and
the stabilizer assembly comprises:
a rail oriented in an axial direction;
a first post slidably couple to the rail, wherein the first shaft is configured to couple to the first post;
a second post slidably coupled to the rail, wherein the second shaft is configured to couple to the second post;
a guide member fixedly coupled to the first post and slidably coupled to the second post; and
a locking assembly disposed on the second post and configured to selectively engage the rail and the guide member;
sliding the first post and the second post in unison in a distal direction along the rail;
actuating the locking assembly to engage the rail and disengage the guide member; and
sliding the first post in the axial direction relative to the second post.

17. The method of claim 16, wherein the method further comprises a step, after sliding the first post in the axial direction relative to the second post, of actuating the locking assembly to disengage the rail and engage the guide member.

18. The method of claim 16, wherein the method further comprises a step, after actuating the locking assembly to disengage the rail and engage the guide member, of disengaging the second shaft from the second post and actuating the second shaft in a proximal direction.

19. The method of claim 18, wherein the method further comprises a step, after actuating the second shaft in the proximal direction, of actuating the second shaft in the distal direction.

20. The method of claim 19, wherein the method further comprises a step, after disengaging the second shaft from the second post and actuating the second shaft in the proximal direction and before actuating the second shaft in the distal direction, of actuating a handle of the delivery apparatus in the proximal direction.
